(19) United States
(12) Patent Application Publication
(10) Pub. No.: US 2014/0221460 A1
(43) Pub. Date: Aug. 7, 2014

(54) METHOD FOR IDENTIFYING MODULATORS OF HCV TRANSLATION OR REPLICATION INVOLVING THE NS5B POLYPEPTIDE AND A PSEUDOKNOT

(71) Applicants: David J. Evans, Coventry (GB); Peter Simmonds, Midlothian (GB)

(72) Inventors: David J. Evans, Coventry (GB); Peter Simmonds, Midlothian (GB)

(73) Assignees: UNIVERSITY COURT OF THE UNIVERSITY OF EDINBURGH, Edinburgh (GB); THE UNIVERSITY OF WARWICK, Coventry (GB)

(21) Appl. No.: 14/239,593

(22) PCT Filed: Aug. 17, 2012
(86) PCT No.: PCT/GB2012/052015

§ 371 Date: Apr. 10, 2014

(30) Foreign Application Priority Data
Aug. 19, 2011 (GB) 1114391.4

(51) Int. Cl. C12Q 1/70; C12N 15/113

(52) U.S. Cl. CPC C12Q 1/707; C12N 15/1131
USPC 514/44 A; 435/5; 435/320.1; 536/24.5; 506/10

(57) ABSTRACT

The invention relates to a method for identifying compounds that act as modulators of hepatitis C (HCV) translation and/or replication, and to compounds identified by this method, and their uses in medicine. The invention also relates to an RNA useful for identifying modulators of HCV translation and/or replication. The invention further relates to a method for producing a replication-competent HCV virus.

### FIELD OF THE INVENTION

The invention relates to a method for identifying compounds that act as modulators of hepatitis C (HCV) translation and/or replication, and to compounds identified by this method and their uses in medicine. The invention also relates to an RNA useful for identifying modulators of HCV translation and/or replication. The invention further relates to a method for producing a replication-competent HCV virus.

### BACKGROUND TO THE INVENTION

HCV is a globally important viral pathogen infecting ˜170 million individuals worldwide. If acute infection is not cleared the virus causes persistent liver disease leading to irreversible cirrhosis and is associated with over 100,000 cases of hepatocellular carcinoma per annum. In the US and Europe, HCV-induced liver disease is the major indication for liver transplantation.

With no current vaccines against HCV, and a level of virus variation that makes the prospect of an effective candidate unlikely, current treatment is restricted to a combination of Ribavirin and pegylated interferon-α. Novel therapies are urgently needed. Reverse genetic approaches to dissect the structure and function of the HCV genome have also been hampered by the limited number of in vitro replication systems available.

There is thus a need for an improved understanding of the molecular biology of HCV to assist identification of novel therapeutic targets.

### SUMMARY OF THE INVENTION

The invention targets molecular interactions involving RNA secondary structures of the HCV virus in order to identify compounds which can modulate HCV translation and/or replication. Modification of RNA secondary structure of the HCV virus also permits production of replication-competent HCV viruses.

The inventors have surprisingly shown that a molecular interaction involving the SL9266/PK pseudoknot, also described herein as SL9266/PK, has effects on HCV translation. This interaction is also dependent on the NS5B polypeptide. Identification of a modulatory effect of a given compound on HCV translation can also identify a modulatory effect on HCV replication.

Thus, the invention provides a screening method for compounds that modulate HCV translation and/or replication. This method utilises components of the above molecular interaction in the form of an RNA comprising the SL9266/PK or a variant thereof, and the NS5B polypeptide. The effects of test compounds on HCV translation are determined in the context of these components. This provides a novel method of drug development which can assist development of therapeutic strategies addressing HCV infection.

Furthermore, the inventors have shown that modification of the stability of RNA secondary structures of the HCV virus, such as the SL9266/PK can have beneficial effects for production of replication-competent HCV. This provides a further benefit in terms of allowing for provision of improved in vitro systems for analysis of HCV.

Accordingly, the invention provides a method for identifying a compound that modulates hepatitis virus C (HCV) translation and/or replication, said method comprising:

(a) contacting an RNA comprising the SL9266/PK pseudoknot or a variant thereof and a translatable reporter coding sequence with the compound in the presence of an NS5B polypeptide or a variant thereof; and

(b) measuring translation of the reporter coding sequence.

In accordance with a preferred aspect of the invention, the method further comprises:

(c) comparing the translation of the reporter coding sequence measured in b) with a control value obtained for an RNA of (a) that has not been contacted with the compound, and thereby determining whether the compound is a modulator of HCV translation and/or replication.

The method of the present invention may be used in a method for identifying a compound that enhances or inhibits repression of HCV translation by the NS5B polypeptide or in a method for identifying a compound that increases or decreases HCV replication, or in a method for identifying a compound suitable for the prevention or treatment of HCV infection.

In another aspect, the present invention provides an RNA comprising the SL9266/PK pseudoknot or a variant thereof and a translatable reporter coding sequence. In a preferred embodiment, the RNA further comprises a translatable NS5B or NS5B variant coding sequence, optionally wherein the reporter coding sequence and NS5B or NS5B variant coding sequence are located on different cistrons.

In accordance with the present invention, a modulator of HCV translation and/or replication identified by the method of any of the present invention may be used in a method of preventing or treating HCV infection in a subject, comprising administering to the subject an effective amount of a modulator of HCV translation and/or replication.

In a further aspect, the present invention provides a method for producing a replication-competent HCV virus, said method comprising:

(a) determining the stability of RNA secondary structures of one or more portions of the genome of the HCV virus;

(b) comparing the stability of said RNA secondary structures with the stability of corresponding structures of the JFH-1 HCV virus; and

(c) introducing mutations into the genome of the HCV virus which stabilise said RNA secondary structures in a similar manner to the corresponding structures of the JFH-1 HCV virus, thereby producing a replication-competent HCV virus.

In another aspect, the invention provides an oligonucleotide comprising 8 to 48 nucleotides in length, wherein the oligonucleotide is substantially complementary to part or all of the region from 9266 to 9314 of HCV. Such oligonucleotides are useful in the treatment or prevention of HCV infection.

### BRIEF DESCRIPTION OF THE FIGURES

FIG. 1 shows analysis of the structure of SL9266/PK in two different in vitro HCV replication systems. (A) Con1b. (B) JFH-1. Key positions are prefixed ˜ to indicate particular nucleotides, and adjacent stem-loops are shown in reverse video and the upstream and ‘kissing loop’ interactions labelled with grey shaded lozenges.

FIG. 2 shows in vitro analysis of SL9266/PK and NS5B-mediated translation feedback. (A) A bicistronic reporter system and capped NS5B mRNA. The asterisk indicates a termination codon engineered into the version of the bicistronic reporter designated NS5B-stop. (B) Luciferase activity normalised to the parental bicistronic reporter as determined in the presence of the reporter listed in the left hand column. C9302A is a point mutation in SL9266/PK. Capped NS5B mRNA was supplemented in trans over a range from 1:1 to 1:10 molar excess.

FIG. 3 shows analysis of the structure of SL9571 in different in vitro replication systems. Each bar represents chemical reactivity of individual nucleotides in the region 9566-9602 with unpaired nucleotides exhibiting greater reactivity. The inverted arrows indicate the inferred duplexed regions of SL9571 and the dotted line the apical loop implicated in ‘kissing loop’ formation with SL9266. (A) Con1b, (B) JFH-1 and (C) JFH-1 with a G9583A mutation which destroys the ‘kissing loop’ interaction.

FIG. 4 shows a schematic diagram and comparison of the interactions of SL9266 with long-range RNA sequences in J6/JFH-1 and Con1b, together with an indication of the influence of specific mutations.

FIGS. 5 and 6 show further schematic analysis of the structures and oligonucleotides directed against SL9266 and SL9571 of Con1b and SL9266 and SL9571 of JFH-1 respectively. HCV sequences complementary to oligonucleotides used in the Examples are illustrated in bold.

FIG. 7 shows the results of assays using the bicistronic reporter to assess the effect of oligonucleotides directed against SL9266 on translation. Lanes 1-5 use the reporter that synthesises NS5B, lanes 6-10 use the reporter that does not synthesise NS5B.

FIG. 8 shows the results of assays to assess the effect of the oligonucleotides directed against SL9266 and SL9571 on replication of a sub-genomic replicon.

FIG. 9 shows the results of assays using the oligonucleotides directed against SL9571 and SL9266 in a virus replication assay.

### DESCRIPTION OF THE SEQUENCES

SEQ ID NO: 1 is the nucleic acid sequence of the NS5B polymerase.

SEQ ID NO: 2 is the amino acid sequence of the NS5B polymerase.

SEQ ID NO: 3 is the nucleic acid sequence of a bicistronic reporter construct

SEQ ID NO: 4 is the nucleic acid sequence of a region of the HCV core protein coding sequence containing a portion of the 5′ HCV IRES.

SEQ ID NO: 5 is the nucleic acid sequence of an oligonucleotide complementary to the SL9571 stem loop.

SEQ ID NO: 6 is the nucleic acid sequence of a Con1b anti-SL9266 LNA oligonucleotide.

SEQ ID NO: 7 is the nucleic acid sequence of a randomised LNA oligonucleotide.

SEQ ID NO: 8 is the nucleic acid sequence of a JFH-1 anti-SL9266 LNA oligonucleotide.

SEQ ID NO: 9 is the nucleic acid sequence of a Con1b/JFH-1 anti-SL9571 LNA oligonucleotide.

SEQ ID NO: 10 is a Con1b nucleic acid sequence.

SEQ ID NO: 11 is the nucleic acid sequence of the SL9266_C oligonucleotide.

SEQ ID NO:12 is the nucleic acid sequence of the SL9266_J oligonucleotide.

SEQ ID NO: 13 is a Con1b SL9571 nucleic acid sequence.

SEQ ID NO: 14 is a JFH-1 SL9266 nucleic acid sequence.

SEQ ID NO: 15 is a JFH-1 SL9266 nucleic acid sequence.

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural referents unless the content clearly dictates otherwise. Thus, for example, reference to “a compound” includes “compounds”, reference to “a polypeptide” includes two or more such polypeptides, and the like. All publications, patents and patent applications cited herein, whether supra or infra, are hereby incorporated by reference in their entirety.

Identification of Compounds that Modulate HCV Translation and/or Replication

The invention provides a method for identifying a compound that acts as a modulator of HCV translation and/or replication. The method involves the contacting of an RNA comprising the SL9266/PK pseudoknot or a variant thereof and a translatable reporter coding sequence with the compound in the presence of an NS5B polypeptide. The method further involves measurement of translation of the reporter coding sequence.

The method may also be used to identify a compound that enhances or inhibits repression of HCV translation by the NS5B polypeptide. The method may further be used to identify a compound that enhances or inhibits the RNA polymerase activity of the NS5B polypeptide or to identify a compound which decreases or increases HCV replication. The method is also preferably used to identify a compound suitable for the prevention or treatment of HCV infection.

RNA

The RNA comprises the SL9266/PK pseudoknot or a variant thereof and a translatable reporter coding sequence. The RNA may be transcribed from a DNA molecule, as described below. The RNA is preferably a single-stranded RNA.

Typically, the RNA is smaller in size than the native HCV genome and does not include the coding region or substantially all of the coding region of the HCV genome. The RNA may lack one or more of, such as two or more, three or more or all of the E1, E2, p7, NS2, NS3, NS4A, NS4B and NS5A coding sequences of the HCV genome. The RNA may lack the coding sequence for the core HCV protein, or only comprise a fragment thereof, as described below. The RNA may lack a full-length NS5B coding sequence, where NS5B is provided in trans. However, the RNA retains sufficient NS5B coding sequence to allow for formation of an SL9266/PK.

The RNA may lack a 5′ non-coding HCV region or may include only a fragment thereof which is sufficient to provide for HCV translation and/or replication. Similarly, the RNA may lack a 3′ non-coding HCV region or may include only a fragment thereof which is sufficient to provide for HCV translation and/or replication. However, the RNA retains sufficient 3′ HCV non-coding region sequence to allow for formation of an SL9266/PK.

The SL9266/PK may thus be only the HCV-derived sequence included in the RNA. However, the RNA preferably comprises the full-length NS5B coding sequence. More preferably, the RNA further comprises a full-length HCV 3′ non-coding region and/or a full-length HCV 5′ non-coding region. Fragments of the HCV 3′ non-coding region and/or the HCV 5′ non-coding region which can provide for HCV translation and/or replication may be used instead of the full length non-coding regions.

Preferably, the 5′HCV IRES or a fragment thereof is included in the RNA. The 5′ HCV IRES comprises four stem-loop structures (domains I-IV) located in the 5′non-coding region and totalling around 340 nucleotides in length, and extends into the polyprotein coding sequence. Conserved structures in the core-coding region are designated as domains V and VI.

It is particularly preferred that the RNA includes a core protein coding sequence or a fragment thereof which includes regions of RNA secondary structure present at the 3′ end of the HCV IRES. This fragment is further described below. Each and every one of the HCV sequences described above are disclosed for use in combination with each other in all possible permutations in the RNA.

The RNA may be of any size. Typically, the RNA is about 15 kb or less in size. The RNA may be about 12 kb or less, about 10 kb or less, about 9 kb or less, about 8 kb or less, about 7 kb or less, about 6 kb or less, about 5 kb or less, about 4 kb or less, about 3 kb or less, about 2.5 kb or less, about 2 kb or less, about 1.5 kb or less, or about 1 kb or less in size. For example, the RNA may be about 1 kb to about 10 kb, about 2 kb to about 10 kb, about 2 kb to about 5 kb, or about 5 kb to about 10 kb in size.

An RNA of a larger size, such as an RNA from about 10 kb to about 15 kb in size is typically used where a full length or substantially full length HCV genome is provided. Smaller RNAs comprise the minimal elements necessary to monitor an effect on HCV translation, i.e. the SL9266/PK or a variant thereof, a reporter coding sequence and optionally an NS5B coding sequence. For example, the SL9266/PK is about 600 nucleotides (nt) in size and a GFP reporter coding sequence may be about 600 nt in size; thus these elements may be provided in an RNA of about 1.2 kb in size. Preferably, the HCV 5′ NCR (347 nt) and a second IRES (perhaps another 350 nt) would be included, providing an RNA of about 1.9 kb in size.

The RNA equivalent to the bicistronic reporter construct of SEQ ID NO: 3 is about 4.7 kb in size and comprises the HCV 5′ non-coding region (7-347 nt), a small region of the HCV core protein coding region (348-395 nt), the luciferase reporter gene (395-2057 nt), a second internal ribosome entry site derived from encephalomyocarditis virus (2076-2676 nt), a V5 tag (2677-2721 nt.), the NS5B protein (2722-4497 nt.) and the 3′ non-coding region of HCV (4498-4728 nt).

The SL9266/PK utilised in the RNA comprises a core RNA stem-loop and sequences 5′ and 3′ to the stem-loop which form a region of extended RNA secondary structure, also described as a pseudoknot. The sequence of the SL9266/PK is derived from the 3′ portion of the NS5B coding sequence and extends into the 3′ non-coding region of the HCV genome.

The HCV 3′ non-coding region is around 200 nucleotides in length and comprises three discrete stem-loops, known as SLI-III, numbered from the 3′ end which forms a structure known as the X-tail. This structure is separated from the HCV coding region by a hypervariable domain and a pyrmidine-rich tract of variable length and sequence. The sequences 5′ proximal to the 3′ NCR encoding the NS5B polypeptide contain five additional phylogenetically conserved RNA stem-loop structures. These are designated, according to the convention described herein as SL9033, SL9132, SL9217, SL9266 and SL9324. SL9266 is predicted to occupy the central position in a cruciform structure involving the adjacent SL9217 (5BSL3.1) and SL9324 (5BSL3.3) stem-loops.

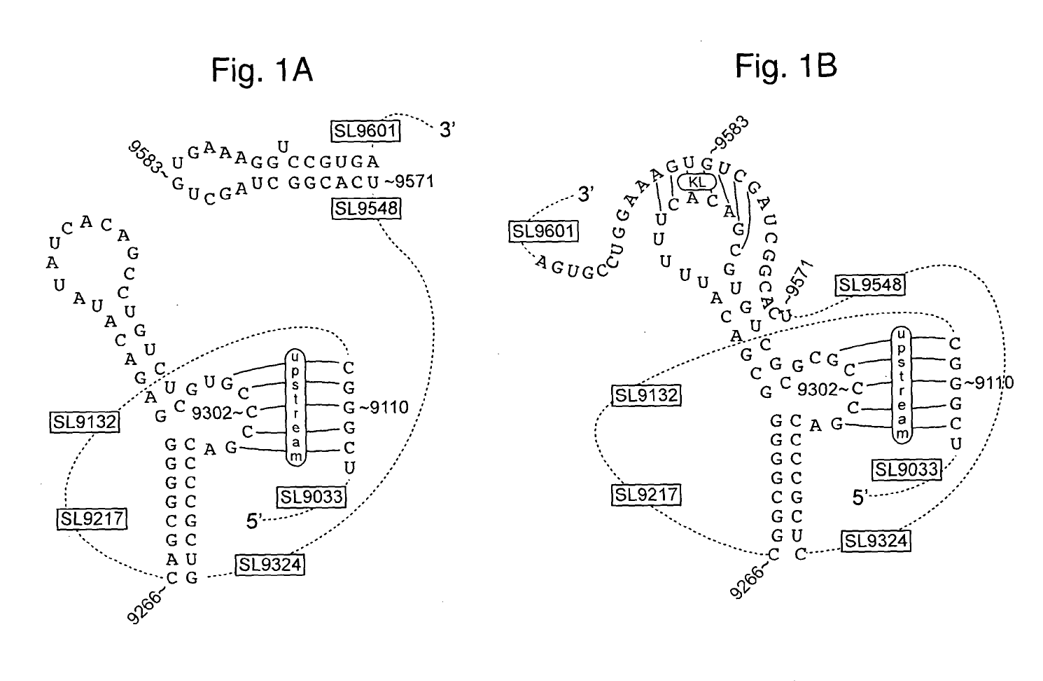

The core RNA stem-loop termed SL9266 is also known in the art as 5BSL3.2 or SL-V. The SL9266 structure comprises an apical loop and two short base paired helices separated by a 3′ subterminal bulge (see FIG. 1). The apical loop and 3′ subterminal bulge loop are involved in upstream and downstream long range RNA-RNA interactions which create a region of extended RNA secondary structure. The long-range interactions occur with sequences around 200 nucleotides upstream and downstream of the SL9266 stem-loop. The structure of the SL9266/PK is reviewed in more detail in Diviney et at (J. Viol (2008) 82, pp 9008-9022).

Provision of an SL9266/PK requires that sufficient sequence of the HCV genome is provided to include the SL9266 stem-loop, and sequences upstream and downstream involved in long range interactions with the stem-loop, and also allowing for folding and stabilisation of the extended region of RNA secondary structure.

A native SL9266/PK used in the RNA typically comprises, consists or consists essentially of the sequence from about nucleotide 9000 to about nucleotide −9650 of the HCV genome. Variants of the SL9266/PK include truncated and modified versions thereof which retain function of the native SL9266/PK, as described below.

The SL9266 nomenclature references the HCV genotype 1a prototype strain H77 22, where the 5′ nucleotide of the SL9266 core RNA stem-loop is at position 9266. Nomenclature of the HCV genome is usefully reviewed in Kuiken, C et at (Hepatology (2006) 44, pp 1355-1361) and Lemon et at (Fields virology 5^{th} Ed. (2007), Hepatitis C virus p 1253-1304).

By definition, the Kuiken paper describes a numbering system that is universal for all HCV genotypes. Sequences that are aligned will always have the same structure in the same place assuming they are phylogenetically conserved. The SL9266/PK is phylogenetically conserved for all HCV genotypes. Accordingly, the skilled person may refer to the above reference sequence in relation to the nucleotide positions given for sequences described herein and extrapolate to the identical position in other genomes and genotypes.

A SL9266/PK used in an RNA according to the invention may be derived from any naturally derived genotype, serotype or isolate or clade of HCV. As is known to the skilled person, HCV viruses occurring in nature may be classified according to various biological systems. The skilled person can provide a sequence corresponding to the SL9266/PK from any naturally derived genotype, serotype or isolate or clade of HCV based on their general knowledge.

HCV genotypes are typically referred to in terms of their genotype. HCV genotypes number from 1 to 11, each has a number of sub-types (a, b, c etc). Representative genotypes and accession numbers include: Genotype 1b (Con1 isolate) AJ238799, and Genotype 2a (JFH-1 isolate) AB047639.

HCV viruses may be referred to in terms of their serotype. A serotype corresponds to a variant subspecies of HCV which owing to its profile of expression of capsid surface antigens has a distinctive reactivity which can be used to distinguish it from other variant subspecies. Typically, a virus having a particular HCV serotype does not efficiently cross-react with neutralising antibodies specific for any other HCV serotype.

HCV viruses may also be referred to in terms of clades or clones. This refers to the phylogenetic relationship of naturally derived HCV viruses, and typically to a phylogenetic group of HCV viruses which can be traced back to a common ancestor, and includes all descendants thereof. Additionally, HCV viruses may be referred to in terms of a specific isolate, i.e. a genetic isolate of a specific HCV virus found in nature. The term genetic isolate describes a population of HCV viruses which has undergone limited genetic mixing with other naturally occurring HCV viruses, thereby defining a recognisably distinct population at a genetic level.

The skilled person can select an appropriate genotype, serotype, clade, clone or isolate of HCV for use in providing an SL9266/PK and any further HCV sequences for an RNA used in the present invention on the basis of their common general knowledge. It should be understood that the invention also encompasses use of an SL9266/PK and any further sequences from an HCV genome of a genotype, serotype, clade, clone or isolate of HCV that may not yet have been identified or characterised.

In addition, the invention encompasses the use of an SL9266/PK from any known in vitro HCV replication systems. These include the sub-genomic replicon (SGR) generated from a HCV genotype 1b consensus sequence Con1b (Lohmann et al, (1999) Science 285, pp 110-113). Another suitable system is the full-length genotype 2a HCV described as JFH-1/HCVcc (Wakita et al, Nat Med (2005) 11, pp 791-796).

The invention also encompasses use of variants of an SL9266/PK. A variant of an SL9266/PK is any sequence derived from an SL9266/PK which includes the SL9266 stem-loop or a variant thereof, and sequences upstream and downstream thereof which allow for folding and stabilisation of the extended region of RNA secondary structure.

As described in Diviney et at supra, the SL9266/PK functions as a cis-acting replication element for HCV. Surprisingly, the present inventors also discovered that the SL9266/PK is necessary for control of HCV translation. In particular, SL9266/PK together with expression of NS5B control the termination of translation, such that replication can start.

Thus, a variant of an SL9266/PK typically comprises the necessary sequences from the NS5B coding sequence and 3′ non coding region of an HCV genome that permit translation from an HCV IRES. Preferably, the variant may comprise the necessary sequences which permit translation of a full-length HCV coding region from an HCV IRES in the context of full-length native 5′ and/or 3′ non-coding HCV regions. The variant may also comprise the necessary sequences from the NS5B coding sequence and 3′ non coding region of an HCV genome that permit replication of an HCV genome in vitro.

Additionally and also unexpectedly, the inventors discovered that the NS5B HCV RNA polymerase can repress HCV translation in the context of the SL9266/PK.

Accordingly, a variant of an SL9266/PK is also described herein as any sequence derived from the NS5B coding region and 3′ non-coding region of an HCV genome which allows for an NS5B polypeptide to repress translation of the reporter coding sequence from the RNA.

It should be understood that a variant SL9266/PK does not need to allow for NS5B polypeptide to repress translation to the same extent as a wildtype native SL9266/PK. A variant of SL9266/PK is any variant that allows for a measurable effect of NS5B on translation which in turn permits identification of the effect of a tested compound on HCV translation and/or replication. A variant of SL9266/PK may translate a reporter coding sequence at 40% or more, typically 50%, 60%, 70%, more preferably 80%, 85%, 90%, 95% or more of the level observed using the native SL9266/PK. This translation efficiency may be observed in the context of a reporter coding sequence operably linked to an HCV IRES.

A variant SL9266/PK may comprise, consist or consist essentially of a sequence of an HCV genome beginning from at least about nucleotide position 9000 or more, such as 9010, 9020, 9030, 9040, 9050, 9060, 9070, 9080, 9090, 9100, 9110 or more, and terminating at at least about nucleotide position 9585, 9590,9595 or 9600 of the HCV genome. Each of the above start positions are disclosed in combination with each of the above termination positions.

It should be understood that a variant SL9266/PK may also comprise internal sequence deletions, substitutions or additions as compared to a native SL9266/PK, provided that these modifications still allow for an NS5B polypeptide to repress translation of the reporter coding sequence from the RNA. The modifications preferably do not alter folding or stabilisation of the structure of the SL9266/PK. Preferably, where an NS5B coding sequence is provided in combination with the SL9266/PK on the RNA, any such modifications do not impair function of the encoded NS5B.

It is also preferred that any such modifications are not made in sequence regions which mediate RNA-RNA interactions present in the SL9266/PK. Thus, deletions, substitutions or additions are preferably not made to the sequence of the stem-loop SL9266, or to sequences upstream or downstream of this stem-loop that interact with the stem-loop. By “upstream”, it is meant that a sequence is 5′ of another sequence, such as 5′ to the stem-loop SL9266 in the HCV genome. By “downstream”, it is meant that a sequence is 3′ of another sequence, such as 3′ to the stem-loop SL9266 in the HCV genome.

The sequences which are typically not modified include sequences upstream of the SL9266 stem-loop around nucleotide 9110 of the HCV genome and sequences downstream of the SL9266 stem-loop around nucleotide 9580 of the HCV genome, in particular regions within these upstream and downstream sequences which interact with and/or are at least partially complementary in sequence to the SL9266 stem-loop as shown for example in FIG. 1.

These interacting sequences are also referred to herein as “upstream SL9266-interacting sequences” and “downstream SL9266-interacting sequences”. The skilled person can select appropriate nucleotide regions comprising such sequences which are able to interact with and stabilise the SL9266 stem-loop.

Typically, the nucleotide region from at least about nucleotide 9266 to at least about nucleotide 9314 of the HCV genome, more preferably the nucleotide region from at least about nucleotide 9250 to at least about nucleotide 9330, is not modified. The nucleotide region from at least about nucleotide 9108 to at least about nucleotide 9112 of the HCV genome, more preferably the nucleotide region from at least about nucleotide 9090 to at least about nucleotide 9130, is also typically not modified. The nucleotide region from at least about nucleotide 9571 to at least about nucleotide 9586 of the HCV genome, more preferably the nucleotide region from at least about nucleotide 9550 to at least about nucleotide 9590, is also typically not modified

However, it is possible to introduce one or more deletions, substitutions or additions in these regions provided that the NS5B polypeptide remains able to repress translation of the reporter coding sequence from the RNA. The selection of suitable modifications to regions such as the SL9266 stem-loop is further described below.

A variant SL9266/PK may also retain only sequence regions equivalent to those described above which mediate the specific RNA-RNA interactions of the native SL9266/PK, provided these regions are spaced at corresponding distances from each other as in the native structure. The selection of spacing is made to allow for a similar folding and conformation of the variant SL9266/PK to the native folding and conformation. The intervening sequences may be randomly selected as any sequences which are able to provide for repression of translation of the reporter coding sequence from the RNA by the NS5B polypeptide.

A variant SL9266/PK may comprise the sequence of SL9266 or a variant thereof, such as the nucleotide sequence from at least about 9266 to at least about nucleotide 9314 of the HCV genome or a variant thereof, located about 150 to about 600 nucleotides 3′ to an upstream SL9266-interacting sequence described herein. The upstream-interacting sequence may be the sequence from at least about nucleotides 9108 to at least about nucleotide 9112 of the HCV genome or a variant thereof. More preferably, the SL9266 or variant thereof is located about 200 to about 500, such as about 200 to about 300 nucleotides 3′ to the upstream SL9266-interacting sequence. The sequence of the SL9266 or variant thereof may be located at about 150, about 170, about 180, about 190, about 200, about 210, about 220, about 230, about 240, or about 250 nucleotides 3′ to the upstream SL9266-interacting sequence.

A variant SL9266/PK may comprise the sequence of SL9266 or a variant thereof, such as the nucleotide sequence from at least about 9266 to at least about nucleotide 9314 of the HCV genome or a variant thereof, located about 150 to about 300 nucleotides 5′ to a downstream SL9266-interacting sequence described herein. The downstream-interacting sequence may be the sequence from at least about nucleotide 9571 to at least about nucleotide 9586 of the HCV genome or a variant thereof. More preferably, the sequence of SL9266 or a variant thereof is located about 200 to about 250 nucleotides 5′ to the downstream SL9266-interacting sequence. The sequence of SL9266 or a variant thereof may be located at about 150, about 170, about 180, about 190, about 200, about 210, about 220, about 230, about 240, or about 250 nucleotides 5′ to the downstream SL9266-interacting sequence.

Reporter and NS5B Coding Sequences

The reporter coding sequence encodes any polypeptide whose presence can be detected in order to serve as a measure of translation activity. The skilled person is able to select suitable reporter coding sequences on the basis of their common general knowledge and as a function of the type of detection method to be utilised to monitor HCV translation.

As described below, preferred methods of detecting translation of a reporter coding sequence translation involve luminescence, fluorescence, or an immunoassay. Thus, a reporter coding sequence may encode a luminescent or fluorescent protein such that the level of translation may be monitored through measurement of a luminescent or fluorescent signal. A suitable example of a luminescent reporter coding sequence is luciferase. Alternatively, a reporter encoding sequence may encode any polypeptide whose presence can be detected immunologically using a suitable antibody.

The reporter coding sequence must be translatable from the RNA. Where the NS5B polypeptide is also encoded on the same RNA, the NS5B coding sequence must also be translatable. Thus, the reporter coding sequence and NS5B coding sequences are positioned in a suitable location on the RNA that allows for access of and activity of components required for translation. The reporter or NS5B coding sequence may be located at the 5′ end of the RNA and include a 5′ cap. The reporter or NS5B coding sequence may be located at an internal site within the sequence of the RNA. In this scenario, the reporter coding sequence is typically operably linked to an internal ribosome entry site (IRES). The IRES for the reporter coding sequence may be of any origin and have any sequence, so long as it provides for translation of the RNA in the context of the SL9266/PK. The skilled person can select a suitable IRES on the basis of their common general knowledge.

Preferably, the IRES for the reporter coding sequence is derived from HCV, i.e. is an HCV IRES. An HCV IRES may be derived from any naturally derived genotype, serotype, isolate or clade of HCV, including those specifically described above. The native HCV IRES is located within the 5′ non-coding region of the HCV genome and extends into the core-protein coding region.

A variant HCV IRES may also be used. A variant HCV IRES is any sequence derived from an HCV IRES which permits translation of a reporter from an RNA of the invention. Variants include truncated forms and sequences having nucleotide substitutions and/or internal deletions or additions. A specific truncated HCV IRES described herein is shown as SEQ ID NO: 4 and contains only the portion of the HCV IRES derived from the core protein coding sequence. This region contains a number of well-defined RNA secondary structures. The above truncated HCV IRES is not functional in permitting translation on its own but may be provided in combination with another means for effecting translation of the reporter coding sequence i.e. a further IRES or a 5′ cap. The above truncated HCV IRES is preferred for inclusion in an RNA of the invention.

The IRES for the NS5B coding sequence can also be provided from any source, but preferably is not derived from HCV. An example of a suitable IRES is an IRES from an EMCV IRES, although an IRES from another source, viral or cellular, could be used instead.

Where the RNA comprises both a reporter coding sequence and an NS5B coding sequence, these sequences are typically provided on different cistrons of the RNA. These two cistrons may be provided in any suitable configuration that allows for translation of the two coding sequences in the context of the SL9266/PK. It is preferred that the reporter coding sequence is 5′ to the NS5B coding sequence. However, the NS5B coding sequence may be 5′ to the reporter coding sequence.

The SL9266/PK or variant thereof is preferably located 3′ to the reporter coding sequence. However, the SL9266/PK may also be located 5′ to the reporter coding sequence provided that the NS5B polypeptide is able to repress translation of the reporter coding sequence from the RNA.

In addition to the SL9266/PK or a variant thereof, the reporter coding sequence and optionally the NS5B coding sequence, the RNA may further comprise any other regulatory sequences which assist translation. For example, the RNA may comprise adjacent to the reporter coding sequence a 5′ UTR or a 3′UTR providing sequences that enhance translation.

NS5B Polypeptide and Variants Thereof.

The method is carried out in the presence of an NS5B polypeptide or a variant thereof. The cDNA sequence for the NS5B RNA polymerase is shown in SEQ ID NO: 1 and encodes the protein shown in SEQ ID NO: 2.

An NS5B polypeptide or variant thereof is any polypeptide which represses translation of a reporter coding sequence from an RNA of the invention. The NS5B polypeptide or variant thereof may further allow for replication of the HCV genome, but this is not essential. The ability of an NS5B polypeptide or variant thereof to repress translation of the reporter coding sequence can be routinely determined by a person skilled in the art, as described below. Preferably the NS5B polypeptide or variant provides similar or higher repression of translation for the reporter coding sequence as compared to the polypeptide of SEQ ID NO: 2.

A variant of SEQ ID NO: 1 or 2 may comprise truncations, mutants or homologues thereof. A variant of SEQ ID NO: 1 may be any transcript variant thereof which encodes a functional NS5B polypeptide.

Any homologues mentioned herein are typically at least 70% homologous to a relevant region of SEQ ID NO: 1 or 2.

Homology can be measured using known methods. For example the UWGCG Package provides the BESTFIT program which can be used to calculate homology (for example used on its default settings) (Devereux et at (1984) Nucleic Acids Research 12, 387-395). The PILEUP and BLAST algorithms can be used to calculate homology or line up sequences (typically on their default settings), for example as described in Altschul S. F. (1993) J Mol Evol 36:290-300; Altschul, S, F et at (1990) J Mol Biol 215:403-10. Software for performing BLAST analyses is publicly available through the National Center for Biotechnology Information (http://www.ncbi.nlm.nih.gov/).

In preferred embodiments, a variant sequence may encode a polypeptide which is at least 55%, 65%, 70%, 75%, 80%, 85%, 90% and more preferably at least 95%, 97% or 99% homologous to a relevant region of SEQ ID NO: 2 over at least 20, preferably at least 30, for instance at least 40, 60, 100, 200, 300, 400 or more contiguous amino acids, or even over the entire sequence of the variant. The relevant region will be one which provides for the functional activity of NS5B in repressing translation of a reporter coding sequence from an RNA of the invention. The region may be a region of NS5B which interacts with nucleolin, p68 (helicase), heIF4All, hPLIC1 (ubiquitin-like) or cyclophilin B.

Alternatively, and preferably the variant sequence may encode a polypeptide having at least 70%, 75%, 80%, 85%, 90% and more preferably at least 95%, 97% or 99% homology to full-length SEQ ID NO: 2 over its entire sequence. Typically the variant sequence differs from the relevant region of SEQ ID NO: 2 by at least, or less than, 2, 5, 10, 20, 40, 50 or 60 mutations (each of which can be substitutions, insertions or deletions).

A variant NS5B polypeptide may have a percentage identity with a particular region of SEQ ID NO: 2 which is the same as any of the specific percentage homology values (i.e. it may have at least 70%, 80% or 90% and more preferably at least 95%, 97% or 99% identity) across any of the lengths of sequence mentioned above.

Variants of SEQ ID NO: 2 also include truncations. Any truncation may be used so long as the variant is still able to repress translation of a reporter coding sequence from an RNA of the invention. Truncations will typically be made to remove sequences that are non-essential for activity in repressing translation and/or do not affect conformation of the folded protein or protein-protein interactions with relevant interacting proteins. Appropriate truncations can routinely be identified by systematic truncation of sequences of varying length from the N- or C-terminus. Preferred truncations are N-terminal and may remove all other sequences except for the catalytic domain.

Variants of SEQ ID NO: 2 further include mutants which have one or more, for example, 2, 3, 4, 5 to 10, 10 to 20, 20 to 40 or more, amino acid insertions, substitutions or deletions with respect to a particular region of SEQ ID NO: 2. Deletions and insertions and substitutions are typically made in regions that are non-essential for activity in repressing translation and/or do not affect conformation of the folded protein.

Substitutions preferably introduce one or more conservative changes, which replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative change may introduce another amino acid that is aromatic or aliphatic in the place of a pre-existing aromatic or aliphatic amino acid.

Conservative amino acid changes are well known in the art and may be selected in accordance with the properties of the 20 main amino acids as defined in Table A below. An example of a typical substitution is mutation of methionine residues of SEQ ID NO:2, such as that at position 2 in the sequence, to alanine so as to prevent undesired internal translation initiation.

Similarly, preferred variants of the polynucleotide sequence of SEQ ID NO: 1 which may be used to provide the NS5B polypeptide or variant thereof include polynucleotides having at least 70%, 75%, 80%, 85%, 90% and more preferably at least 95%, 97% or 99% homology to a relevant region of SEQ ID NO: 1. Preferably the variant displays these levels of homology to full-length SEQ ID NO: 1 over its entire sequence.

TABLE A
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, | Pro | hydrophobic, neutral |
| | charged (−) | | |
| Glu | polar, hydrophilic, | Gln | polar, hydrophilic, neutral |
| | charged (−) | | |
| Phe | aromatic, hydrophobic, | Arg | polar, hydrophilic, |
| | neutral | | charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, |
| | | | neutral |
| Lys | polar, hydrophilic, charged(+) | Trp | aromatic, hydrophobic, |
| | | | neutral |
| Leu | aliphatic, hydrophobic, neutral | Tyr | aromatic, polar, |
| | | | hydrophobic |

Compound to be Tested

The compounds tested may be enhancers or inhibitors of HCV translation. An enhancer of HCV translation increases the translation of a reporter coding sequence. An inhibitor of HCV translation decreases the translation of a reporter coding sequence. An enhancer of HCV translation may inhibit replication of HCV, such as by decreasing HCV replication or rendering the HCV replication-incompetent. An inhibitor of HCV translation may activate or increase replication of HCV.

A modulator of HCV translation may increase or decrease translation of a reporter coding sequence by any mechanism. Likewise, a modulator of HCV replication may inhibit, activate or increase HCV replication by any mechanism.

For instance, a modulator of HCV translation and/or replication may act directly by binding to NS5B polypeptide. The NS5B polypeptide is an RNA polymerase. Thus, the modulator may bind directly at the enzyme active site or may bind at another site and exert allosteric effects on enzyme function. The modulator may affect an interaction between the NS5B polypeptide and a region of the HCV RNA genome, such as a region of RNA secondary structure. A modulator of HCV translation and/or replication may bind directly to the SL9266/PK or to another element of RNA secondary structure of an HCV genome.

A modulator of HCV translation and/or replication may also act indirectly on the molecular interaction between the SL9266/PK and the NS5B polypeptide. It may act on an additional component of an NS5B-containing protein complex. It may also act on other cellular factors necessary for the repressive effect of the NS5B polypeptide on HCV translation. It may also have effects on activation of NS5B polymerase activity, for example by acting via secondary messenger systems. A modulator of HCV translation and/or replication may also act at the level of NS5B translation so as to increase or decrease NS5B mRNA or protein levels. It may also act to regulate the stability of the expressed mRNA or protein.

Any compound(s) can be used in the method of the invention. The compound(s) are preferably ones that are suspected of modulating HCV translation and/or replication.

The compound(s) can be provided in any suitable form, as described below.

The compound(s) may be any chemical compound(s) used in drug screening programmes. They may be natural or synthetic. Extracts of plants which contain several characterised or uncharacterised components may also be used. Typically, organic molecules will be screened, preferably small organic molecules which have a molecular weight of from 50 to 2500 Daltons. Compounds can be biomolecules including peptide and peptide mimetics, oligonucleotides, saccharides, fatty acids, steroids, purines, pyrimidines, derivatives, structural analogs or combinations thereof. Candidate compounds may be obtained from a wide variety of sources including libraries of synthetic or natural substances. Known pharmacological agents may be subjected to directed or random chemical modifications, such as acylation, alkylation, esterification, amidification, etc. to produce structural analogs. The compound(s) may be the product(s) of a combinatorial library such as are now well known in the art (see e.g. Newton (1997) Expert Opinion Therapeutic Patents, 7(10): 1183-1194). Natural product libraries, such as display (e.g. phage display libraries), may also be used.

Antibodies directed to a component of the molecular interaction regulating translation and involving the SL9266/PK pseudoknot, such as the NS5B polypeptide or the SL9266/PK, are another class of suitable compounds. For example, monoclonal and polyclonal antibodies, single chain antibodies, chimeric antibodies, CDR-grafted antibodies and humanized antibodies may be used. The antibody may be an intact immunoglobulin molecule or a fragment thereof such as a Fab, F(ab′)₂ or Fv fragment. Candidate inhibitor antibodies may be characterised and their binding regions determined to provide single chain antibodies and fragments thereof which are responsible for disrupting the relevant interaction.

A suitable antibody may bind to either the NS5B polypeptide or the SL9266/PK, and thereby prevent or block a direct or indirect interaction between these components. Antibodies may be raised against specific epitopes of the NS5B polypeptide or the SL9266/PK.

RNA interference may also be used to downregulate expression of candidate genes of interest for screening for their effect on HCV translation and/or replication, such as by use of siRNA libraries.

Additionally, oligonucleotides which bind to SL9266 (or interacting sequences) and so prevent SL9266 folding, long range interactions and function are another class of suitable compounds. For example, LNA (Locked Nucleic Acids) oligonucleotides may be used (see http://en.wikipedia.org/wiki/Locked_nucleic_acid).

Assay Conditions and Measurement of Translation/Replication

The methods of the invention allow the screening of one or more compounds for their ability to act as modulator of HCV translation and/or replication. The methods are preferably carried out in vitro or ex vivo.

The method can also be used to confirm the effects of a modulator of HCV translation and/or replication identified by any other means. Thus, for example, whether

a known modulator of HCV translation and/or replication enhances or inhibits repression of HCV translation by the NS5B polypeptide. Also, for example, whether a known modulator of HCV translation and/or replication enhances or inhibits activation of HCV replication by the NS5B polypeptide.

Techniques for determining the effect of compound(s) on the translation of a reporter coding sequence are well known in the art. Any of those techniques may be used in accordance with the invention.

The method may be carried out in any suitable assay system permitting measurement of translation of a reporter coding sequence. The method may be carried out in vitro, such as in a cell-free system or alternatively in a cell-based system.

Preferred in vitro translation systems utilise cell extracts which provide components necessary for the process of translation. These typically include macromolecular components such as ribosomes, tRNAs, aminoacyl tRNA synthetases, initiation, elongation and termination factors. The cell extracts may be of any origin provided they allow for translation of the reporter coding sequence. Suitable cell extracts may be obtained from reticulocytes, such as rabbit reticulocytes, wheat germ and bacterial extracts, such as E. coli extracts. The cell extracts are suitably supplemented with additional components required for translation, such as amino acids, nucleotide triphosphate energy sources and other co-factors. The skilled person is familiar with the use of such systems.

In preferred embodiments, the RNA is added to the in vitro translation system directly in RNA form. Alternatively, a coupled or linked transcription/translation system may be utilised in which a DNA construct encoding the RNA is first transcribed prior to translation of the resulting RNA.

In addition to the use of cell extracts to provide translation machinery, cytoplasmic extracts from other cells may be included in the in vitro assay in order to investigate the role of further components in effects on HCV translation and/or replication. An example of suitable cell extracts include Huh-7 or Huh7.5 cell extracts.

Cell-based methods of the invention typically require the transfection of a reporter construct into a suitable cell line. Typically, the reporter construct is transfected in RNA form. However, it is also possible to transfect a DNA construct encoding the RNA into the cell line which is then transcribed into RNA. Such a DNA construct may also be provided integrated in the genome of the cell. Similarly, where NS5B is provided in trans, an RNA or DNA construct comprising an NS5B coding sequence may also be transfected into the cell. The above DNA construct may also be provided integrated in the genome of the cell. Expression of the reporter coding sequence and/or the NS5B coding sequence may be transient or stable, inducible or constitutive. The compound may also be expressed in the cell or added exogenously.

The method can be carried out using any NS5B polypeptide in any form. Suitable NS5B polypeptides are discussed in more detail below. Typically, only one NS5B polypeptide is used. However, in some embodiments, it is possible to use two or more, such as 3, 4 or 5 or more, different NS5B polypeptides.

The NS5B polypeptide may be provided in trans or may be expressed from the same construct as the reporter coding sequence. Where the NS5B polypeptide is provided in trans, it may be expressed from an RNA or DNA construct or provided directly in polypeptide form.

Where the NS5B polypeptide is expressed from a DNA construct, for example by transfection into a cell, the cell which is transfected suitably expresses an RNA polymerase capable of transcribing the NS5B coding sequence. The RNA polymerase may for example be a T7 RNA polymerase. Alternatively, a ubiquitous promoter allowing transcription by any RNA polymerase, such as a cytomegalovirus promoter, may be operably linked to the NS5B coding sequence.

The NS5B polypeptide can be in solution. The solution may comprise a purified or substantially purified recombinant NS5B polypeptide in a suitable buffer. Such buffers are known in the art. Alternatively, the solution may be a culture medium or a cell lysate from a cell culture expressing a NS5B polypeptide. The NS5B polypeptide may also be immobilised on a platform or surface. Suitable platforms or surfaces are known in the art. An example is a standard 96 or 384 well plate.

If the contacting with a compound takes place in solution or on a surface or platform, the method is carried out under conditions that allow NS5B to function, and in particular to control translation. Suitable conditions include, but are not limited to 20 mM Tri-HCl (pH 7.5), 5 mM MgCl₂, 1 mM dithiothreitol, 25 mM KCl, 1 mM EDTA with incubation temperatures between 20° C. and 37° C.

The NS5B polypeptide may be contacted first with compound and then introduced into the presence of the RNA. This type of pre-incubation may be necessary to allow sufficient time for a compound to have an effect on NS5B activity. Alternatively, the compound may be introduced into the presence of the NS5B polypeptide and the RNA at the same time. Contacting with the compound is carried out for a sufficient period to allow for HCV translation to be measured by the methods described below.

Where the NS5B polypeptide and the RNA are expressed in a cell or cell culture, the method is carried out under conditions that maintain viability of the cell or the cell culture. Suitable conditions include, but are not limited to, a humidified atmosphere of 5% CO2 at 37° C. in appropriate culture media. Suitable cells include Huh-7 or Huh7.5 cells, HepG2, HeLa, 293, NIH3T3 and CHO cells.

The method of the invention can be carried out in a single reaction (i.e. one which contains at least one compound, an NS5B polypeptide and RNA). For instance, the method of the invention can be used to identify whether or not a single individual compound is a modulator of HCV translation and/or replication.

However, as will be appreciated, particularly for in vitro translation systems, the method of the invention is preferably carried out in multiple simultaneous or concurrent reactions, such as 5, 10, 15, 20, 30, 40, 50, 100, 150, 200 or more simultaneous or concurrent reactions. Each reaction contains at least one compound, at least one NS5B polypeptide and at least one RNA. This allows a variety of aspects of modulation of HCV translation and/or replication to be investigated.

Preferably, the method of the invention involves simultaneously or concurrently identifying multiple compounds that modulate HCV translation and/or replication. In other words, the method of the invention may involve high-throughput screening of more than one compound. High-throughput screening is typically carried out using 5, 10, 15, 20, 30, 40, 50, 100, 150, 200 or more different compounds. Typically, each compound is screened in a different reaction. However, two or more compounds may be assayed in the same reaction.

The method of the invention can be used to identify the concentration at which a compound optimally modulates HCV translation and/or replication. In such an embodiment, multiple reactions are simultaneously or concurrently carried out using different concentrations of the compound in each reaction.

Multiple reactions can be carried out in the wells of a flat plate. The wells typically have a capacity of from about 25 μl to about 250 μl, from about 30 μl to about 200 μl, from about 40 μl to about 150 μl or from about 50 to 100 μl. 96 or 384 reactions may be simultaneously or concurrently carried out in the wells of a standard 96 or 384 well plate. Binding proteins or antibodies may be immobilised on a surface of one or more, preferably all, of the wells where required. These can be used to immobilise the NS5B polypeptide to the surface of the wells.

Where multiple reactions are performed, each reaction will typically be carried out under a set of similar conditions to allow for comparison of results obtained. Suitable conditions are set out above. As appropriate, each reaction is also typically carried out using the same molar concentration or input amount of the reaction constituents, namely the compound, the RNA and/or the NS5B polypeptide, to allow for comparison of results obtained. Suitable levels of RNA may be 0.1 to 10 micrograms. Where NS5B is expressed in trans, the NS5B-encoding RNA may typically be at a 0.1-10 fold molar excess compared to the RNA comprising the reporter coding sequence.

The concentration of the compound to be contacted will vary depending on the nature of the compound. A person skilled in the art can determine an appropriate concentration. Typically, concentrations of from about 0.01 to 100 nM of the compound may be used, for example from 0.1 to 10 nM.

Where cells or cell cultures are used, each reaction typically involves the same number of cells. For instance, cells are typically seeded with approximately the same number of cells in each well of a plate, and each reaction is performed after the same time period. Typically 3-5×10⁴ cells are seeded per well of a 96-well plate.

For each of the embodiments discussed above, the precise conditions used in the assay may vary. Experimental conditions may be optimised as a matter of routine by the person skilled in the art on the basis of their general knowledge to improve sensitivity and reliability of the method of the invention.

In order to allow for a determination of whether or not the compound is a modulator of HCV translation and/or replication, a comparison is made with a control value. The value for translation of the reporter coding sequence obtained following contacting of NS5B polypeptide with the compound and the RNA is compared with the control value. The control value is the reporter translation activity observed under conditions where the NS5B polypeptide has been contacted with the RNA, but has not been contacted with the compound. Preferably, the conditions are otherwise identical to those used to obtain the reporter translation value following contacting with the compound. Following the comparison with the control value, the effect of the compound may be identified in terms of an increase in reporter coding sequence translation or a decrease in reporter coding sequence translation with respect to the control value. An increase is indicative of an enhancer. A decrease is indicative of an inhibitor.

Preferably, the control value is obtained while carrying out the method of the invention. For example, a control reaction is performed at the same time as reaction(s) where the NS5B polypeptide is contacted with the RNA and the compound. This ensures that the control value is obtained under the same conditions as the reporter coding sequence translation value measured following contacting of NS5B polypeptide with the RNA and the compound.

The control value can also be obtained separately from the method of the invention. For instance, the control value may be obtained beforehand and recorded, for instance on a computer. The control value may be used for multiple repetitions of the method. The control value can be derived from more than one control reaction. For instance, the control value may be the arithmetic mean of the measurement obtained from several, such as 2, 5, 10, 15 or more, control reactions. In order to allow for an effective comparison, the control value has the same units as the measurement in the test sample with which it is being compared. A person skilled in the art is capable of obtaining such a value.

The type of control value referred to above is commonly known in the art as a “negative control”. The method of the invention can also be carried out in conjunction with one or more positive controls for modulation of HCV translation and/or replication. This involves carrying out reactions using one or more compounds which are known modulation of HCV translation and/or replication. A positive control allows for validation of the measurement of the reporter coding sequence translation activity that is used in the method of the invention. For instance, this may be useful to allow comparison of results from different cell types. A positive control also allows the extent to which the compound modulates HCV translation and/or replication to be determined. An example of a suitable positive control is Ribavirin, an inhibitor of HCV replication.

The incubation period of the reaction constituents prior to measurement of reporter coding sequence translation activity will be selected on the basis of the time required to generate a signal of appropriate strength. Measurement of reporter coding sequence translation can be performed at one or more timepoints following contacting with the test compound. This may allow for a determination of the duration and stability of the effect of the compound.

Techniques for measuring translation of a reporter coding sequence are well known in the art. Any suitable technique may be used. Preferred methods of measuring reporter coding sequence translation involve luminescence, fluorescence, or an immunoassay. For example, a reporter coding sequence may encode a luminescent or fluorescent protein such that the level of translation may be monitored through measurement of a luminescent or fluorescent signal. A suitable example of a luminescent reporter coding sequence is luciferase.

Measuring levels of translated protein using an immunoassay is also well known in the art. Any suitable immunoassay which allows for detection of a reporter coding sequence by an antibody may be used. Any suitable commercially available antibody for a given target may be used. An example of a suitable immunoassay is Enzyme-Linked ImmunoSorbent Assay (ELISA). In some embodiments, the ELISA assay may be performed in flat plates where wells are coated with binding proteins or antibodies which can bind and allow for detection of the translated reporter polypeptide. Other types of immunoassay include immunoprecipitation and Western blotting.

Whilst immunoassays are preferred, any other high-affinity ligand-receptor interaction, such as streptavidin-biotin, could be used to measure translation activity.

As described herein, the repression of HCV translation by the NS5B polymerase is in addition to its known role in providing for HCV replication. Thus, the identification of a compound which modulates HCV translation in the context of the method of the invention may also permit identification of an additional effect of that compound on HCV replication.

The method of the invention may therefore further comprise a step of measuring HCV replication. Preferably, HCV replication is measured in the context of an RNA comprising a full HCV genome or a replication-competent variant thereof. For example, the RNA may comprise the sequence of the SGR or of JFH-1 (HCVcc). However, any suitable system allowing for measurement of an effect at at least one stage of the HCV replication cycle may be utilised. Thus, an effect on HCV replication may be measured without a requirement for a full replication cycle and without any requirement for packaging of the replicated genome.

HCV replication may be measured at the genetic level by incorporation of a reporter gene into a sub-genomic replicon which lacks the structural proteins of the HCV virus—core, E1 and E2. It is also possible to measure replication of a full length genome by incorporation of a reporter—such as luciferase—in-frame within the coding region. HCV replication may also be measured at the level of production of infectious virus particles. A replication-competent HCV genotype would be used for such a measurement, such as the JFH-1 virus.

### Products of the Invention

The invention also provides an RNA comprising the SL9266/PK pseudoknot or a variant thereof and a translatable reporter coding sequence as a product per se. The features of this RNA are as described above in relation to the RNA used in the methods of the invention.

The invention also provides a kit that may be used to carry out the screening method of the invention. The kit typically comprises the RNA or means for expression of the RNA, means for provision of an NS5B polypeptide or variant thereof and optionally instructions to enable the kit to be used in the method of the invention. The means for expression of the RNA may be a DNA construct encoding the RNA, such as a plasmid. The means for provision of the NS5B polypeptide or variant thereof may be included as part of the RNA or as part of the same DNA construct in the situation where the RNA further comprises an NS5B coding sequence. Alternatively, the kit may comprise a second DNA construct encoding an NS5B polypeptide or a variant thereof.

The kit suitably further comprises components necessary for the process of translation. These may be provided in the form of cell extracts as described above. The cell extracts may typically include macromolecular components such as ribosomes, tRNAs, aminoacyl tRNA synthetases, initiation, elongation and termination factors. The kit may include additional components required for translation, such as amino acids, nucleotide triphosphate energy sources and other co-factors.

The kit may additionally comprise one or more other reagents or instruments which enable any of the embodiments of the method mentioned above to be carried out. Such reagents or instruments include one or more of the following: suitable buffer(s) (aqueous solutions), antibodies conjugated to detection moieties, substrates for enzymatically active tags, means to obtain a sample from a subject (such as a vessel or an instrument comprising a needle), means to measure translation of a reporter coding sequence and/or expression or cell culture apparatus. Reagents may be present in the kit in a dry state such that a fluid sample resuspends the reagents.

The kit may also, optionally, comprise instructions to enable the kit to be used in the method of the invention.

The invention further provides a modulator of HCV translation and/or replication identified by the method of the invention as a product per se. These products are described above in the section relating to compounds to be tested.

Preferred modulators of HCV translation and/or replication of the invention include, but are not limited to small organic molecules which have a molecular weight of from 50 to 2500 Daltons, and antibodies.

Modulators of HCV translation and/or replication may be provided isolated and/or purified from their natural environment, in substantially pure or homogeneous form, or free or substantially free of other materials from their source or origin. Where used herein, the term “isolated” encompasses all of these possibilities. They may optionally be labelled or conjugated to other compounds.

Modulators of HCV translation and/or replication can be formulated into pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

For delayed release, the modulators of HCV translation and/or replication may be included in a pharmaceutical composition which is formulated for slow release, such as in microcapsules formed from biocompatible polymers or in liposomal carrier systems according to methods known in the art.

The dose of a modulator of HCV translation and/or replication may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific modulator, the age, weight and conditions of the subject to be treated and the frequency and route of administration. Preferably, daily dosage levels are from 5 mg to 2 g. That dose may be provided as a single dose or may be provided as multiple doses, for example taken at regular intervals, for example 2, 3 or 4 doses administered daily.

### Oligonucleotide Inhibitors

One aspect of the present invention relates to oligonucleotides, having complementarity to SL9266, which may be used for the inhibition of HCV replication or translation. Such oligonucleotides may be useful in the treatment of HCV infection. Typically, such oligonucleotides are provided as single-stranded nucleic acids having phosphodiester, 2′O-methyl, 2′ methoxy-ethyl, phosphoramidate, methylphosphonate, and/or phosphorothioate backbone chemistry. Typically, the oligonucleotides are provided as DNA molecules, having modified chemistry at one or more positions to increase their stability. Typically, locked nucleic acids (LNA) may be provided.

The oligonucleotides for use in accordance with the present invention are substantially complementary to part or all of the SL9266, and interfere with the formation of SL9266, the interactions of this region with other regions of the HCV genome, and/or other interacting proteins. Such oligonucleotides interfere with HCV translation and/or replication. Such oligonucleotides may interfere with one or more of the processes involved in translation and/or replication such that translation and/or replication of the viral genome is reduced. Levels of viral translation and/or replication may be reduced by 10%, 20%, 30%, 40%, 50%, 60%, 70%, 80%, 90% up to 100%. Typically, such oligonucleotides are complementary to part or all of the region from 9266 to 9314. Typically, the oligonucleotide is complementary to a region from 8 to 48 nucleotides within this region, say between 10 and 30 nucleotides, such as between 10 and 25 nucleotides. The oligonucleotides are typically 8 to 30 bases in length, such as 10 to 25 nucleotides in length.

The oligonucleotides are complementary to part or all of the SL9266 stem loop. Typically, oligonucleotides are provided which are 100% complementary. However, lower levels of complementarity may also be acceptable, such as 95%, 90%, 85% or 80%. An oligonucleotide may therefore have 1, 2, 3, 4 up to 5 mismatches across a region of 10, 15, 20, 25 or 30 nucleotides in the SL9266 stem loop. Preferably 100% complementarity is present at positions in part or all of the stem loop that are conserved across HCV genotypes The oligonucleotides can be provided to have complementarity to part or all of the stem portion, part or all of the loop portion or part or all of the stem and loop portion of the SL9266.

An exemplary oligonucleotide has the sequence

TCACGGACCTTTCACAGC.

### Method of Treatment and Medical Use

The invention also provides a method of preventing or treating HCV infection in a subject, comprising administering to the subject an effective amount of a modulator of HCV translation and/or replication identified in accordance with the invention, or an oligonucleotide inhibitor as described above.

The invention also provides a modulator of HCV translation and/or replication identified in accordance with the invention, or an oligonucleotide inhibitor of the invention for use in a method of preventing or treating HCV infection. The invention further provides use of a modulator of HCV translation and/or replication identified in accordance with the invention, or an oligonucleotide inhibitor of the invention in the manufacture of a medicament for preventing or treating HCV infection.

In all these embodiments, the modulator of HCV translation and/or replication or oligonucleotide inhibitor may be administered in order to prevent the onset of one or more symptoms of HCV infection. In this embodiment, the subject can be asymptomatic. The subject may have a predisposition to infection by HCV. A prophylactically effective amount of the modulator of HCV translation and/or replication, or the oligonucleotide is administered to such a subject. A prophylactically effective amount is an amount which prevents the onset of one or more symptoms of HCV infection. The modulator, or oligonucleotide inhibitor may be used to prevent liver disease caused by HCV infection or to prevent hepatocellular carcinoma.

Alternatively, the modulator of HCV translation and/or replication, or oligonucleotide inhibitor may be administered once the symptoms of HCV infection have appeared in a subject i.e. to cure an existing HCV infection. A therapeutically effective amount of the modulator, or oligonucleotide is administered to such a subject. A therapeutically effective amount is an amount which is effective to ameliorate one or more symptoms of HCV infection. Typically, such an amount reduces the HCV infection or viral titre in the subject.

Animal models in which an HCV infection has been established or can be implemented can also be used to identify modulators of HCV translation and/or replication suitable for use in the methods of treatment and medical uses of the invention.

### Method for Producing a Replication-Competent HCV Virus

The invention further provides a method for producing a replication-competent virus. The method comprises:

(a) determining the stability of RNA secondary structures of one or more portions of the genome of the HCV virus;

(b) comparing the stability of said RNA secondary structures with the stability of corresponding structures of the JFH-1 HCV virus; and

(c) introducing mutations into the genome of the HCV virus which stabilise said RNA secondary structures in a similar manner to the corresponding structures of the JFH-1 HCV virus, thereby producing a replication-competent HCV virus.

The inventors have surprisingly identified that the replication—competence of the JFH-1 (HCVcc) HCV virus is associated with the particular conformation and stability of RNA secondary structures of JFH-1 as compared to other HCV genomic constructs, such as the genotype 1b Con1b SGR system and the genotype 1a H77 cDNA. Accordingly, the replication-competence of an HCV virus may be improved by stabilising RNA secondary structures of the virus with an analogous folding and conformation to that observed in JFH-1.

The RNA secondary structures to be stabilised may be any regions of RNA secondary structure of the HCV virus whose stability and conformation have an influence on the replication-competence of HCV. The RNA secondary structures may be located in the 5′ or 3′ non-coding region of the HCV genome, or in the coding region of the HCV genome and may overlap one or more of these regions of the HCV genome.

Preferably, the RNA secondary structures to be stabilised comprise the SL9266/PK pseudoknot. As described herein, the inventors have specifically identified significant differences in the structure and stability of SL9266/PK between the JFH-1 and Con1b SGR systems. These differences are believed to be associated with the weaker replication phenotype of the Con1b-SGR system.

In order to determine the stability of regions of the HCV genome which may be potentially modified to provide for RNA secondary structures having similar stability and conformation to corresponding structures of the JFH-1 HCV virus, the skilled person may use any method known in the art suitable for determination of the existence of regions of RNA secondary structure. A particularly suitable method is SHAPE (Selective 2′ hydroxyl acylation analysed by primer extension). In this method, the existence of base-paired regions can be inferred from the lack of formation of adducts of reagents such as NMIA (N-methylisotoic acid) with 2′ hydroxyl groups of RNA in the relevant region.

Alternative methods to determine the structure and stability of the RNA elements include conducting SHAPE using different chemical modifiers such as 1-methyl-7-nitroisatoic anhydride, chemical mapping using hydroxyl radicals or dimethyl sulphate, use of specific ribonucleases such as V1 or T1 ribonuclease, or nuclear magnetic resonance spectroscopy.

The stability of the regions of RNA secondary structure is then compared to the stability of corresponding regions of the JFH-1 virus. The stability may be determined in terms of numbers of hydrogen bonds within a relevant secondary structure or in terms of overall stability mediated by all relevant molecular interactions, including stacking energies, non-Watson Crick basepairing and opening or closing basepairs. The stability may be calculated thermodynamically. For example, a suitable measure of stability is RNA free energy. The RNA free energy may be calculated by methods known in the art such as Mfold and UNAfold. Suitable methods are described in the following references:
Markham, N. R. & Zuker, M. (2005) DINAMelt web server for nucleic acid melting prediction. Nucleic Acids Res., 33, W577-W581.
Markham, N. R. & Zuker, M. (2008) UNAFold: software for nucleic acid folding and hybriziation. In Keith, J. M., editor, Bioinformatics, Volume II. Structure, Function and Applications, number 453 in Methods in Molecular Biology, chapter 1, pages 3-31. Humana Press, Totowa, N.J. ISBN 978-1-60327-428-9.
M. Zuker. Mfold web server for nucleic acid folding and hybridization prediction. Nucleic Acids Res. 31 (13), 3406-3415, 2003.

The thermodynamic stability of one or more regions of RNA secondary structure across the genome of an HCV virus of interest may be systematically analysed by computational methods.

The method then comprises the introduction of mutations to provide for a comparable stability of the RNA secondary structures to the corresponding structures of the JFH-1 virus. Such mutations may be selected from nucleotide substitutions, additions and deletions. Preferably, where the mutations are introduced into a coding region of the genome of the HCV virus, in particular the NS5B coding sequence, they do not impair function of the encoded protein.

A comparable stability is preferably characterised by the existence of about the same number of hydrogen bonds within each relevant secondary structure as compared to the corresponding structure in JFH-1. For example, where the relevant structure is the SL9266 stem-loop duplex of the HCV virus, a comparable stability may comprise the existence of about the same number of hydrogen bonds as observed in the upper and lower duplex of the SL9266 stemloop in the JFH-1 virus. A comparable stability may also be exhibited in thermodynamic terms as a comparable RNA free energy.

A comparable stability may also or alternatively be characterised by considering the relative stability of the upper and lower duplexes. In particular, the balance between the upper and lower duplexes of JFH-1 contribute to replication competence and allow the region to be independent of the influence of the upstream interaction. In contrast, Con1b has a bigger difference in energy between the upper and lower duplexed regions and is therefore predicted to be less stable. Thus, the present invention encompasses modifications which lead to a smaller difference in energy between the upper and lower duplexed regions, which in turn reduce the requirement for stabilisation from upstream sequences, such as the conserved sequence around nucleotide 9110.

The method of the invention has particular application to the SL9266/PK of HCV.

The mutations may be made in any part of the SL9266/PK which has a role in stabilising the folded conformation of SL9266/PK in JFH-1, but are typically made in the region adjacent to and comprising the SL9266, and the upstream and downstream SL9266-interacting regions described above. The nucleotide positions of these regions in the HCV genome may be as defined above in relation to the SL9266/PK provided in the RNA used for identification of modulators of HCV translation and/or replication.

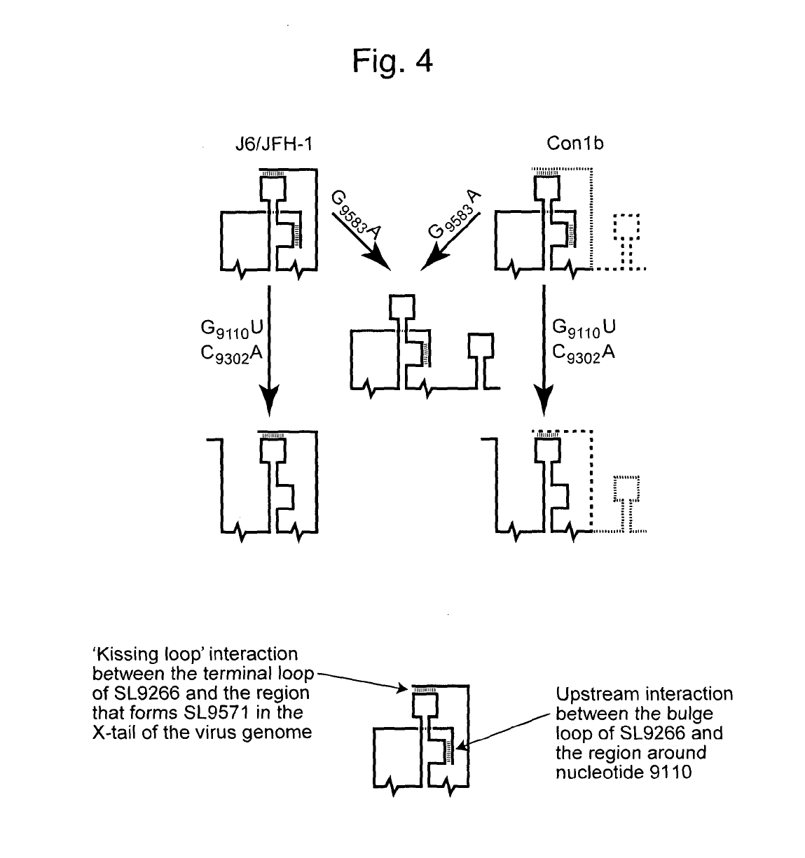

The structure of the SL9266/PK in JFH-1, as shown in FIG. 1 and FIG. 4, is characterised by the existence of interactions between two loop regions of the SL9266 stem-loop with upstream and downstream sequences. The apical loop of SL9266 interacts with the apical loop of stem-loop SL9571 located near the 3′ terminus of the 3′ noncoding region of the HCV genome in a “kissing loop” interaction. The 3′ subterminal bulge loop of SL9266 interacts with an unstructured region centred on nucleotide 9110. These interacting regions are discussed in more detail above. In addition, the SL9266 has upper and lower duplexes separated by the 3′ subterminal bulge.

The stability of the upper duplex of the SL9266 stem-loop adjacent to the apical loop was surprisingly found to be significantly increased in the JFH-1 SL9266/PK as compared to the Con1b SGR. Additionally, the lower duplex at the base of the stem-loop and below the 3′ subterminal bulge was found to have lower stability in JFH-1 as compared to Con1b.

These relative changes in stability have the effect that the upper and lower duplex have more similar stability in JFH-1. These changes in stability of the duplexes are thought to underlie the formation of the kissing loop interaction described above which is observed in JFH-1, but not Con1b. The changes in duplex stability may also destabilise the upstream interaction described above involving the 3′ subterminal bulge loop in JFH-1 as compared to Con1b. More generally, the differences give rise to the specific conformation and stability of the SL9266/PK in the replication-competent JFH-1 system.

Accordingly, the invention provides for the recapitulation of the replication-competent conformation of the SL9266/PK of JFH-1 in an HCV virus of interest by the introduction of mutations into the genome of the HCV virus which stabilise the SL9266/PK in a similar manner to the SL9266/PK of the JFH-1 HCV virus.

Such mutations preferably produce an SL9266/PK which is characterised by having an interaction between the apical loop of SL9266 and a downstream apical loop adjacent to the 3′ terminus of the HCV genome, also described as SL9571. This interaction is also described herein as a “kissing loop” interaction. More preferably, the mutations produce a “kissing loop” interaction having a similar stability to that observed in the JFH-1 virus. The interacting elements preferably also show a similar conformation and folding to that of the JFH-1 virus.

Such mutations also preferably produce an interaction between the 3′ subterminal bulge loop of SL9266 and an upstream region centred around about nucleotide 9110 of the HCV genome which has a similar stability to that observed in the JFH-1 virus. The interacting elements preferably also show a similar conformation and folding to that of the JFH-1 virus.

It is also particularly preferred that such mutations give rise to a similar stability for both the upper and lower duplex of the SL9266 stem-loop in the HCV virus of interest, optimally a comparable relative stability to that observed for the upper and lower duplexes of SL9266 in JFH-1.

The mutations have the effect of producing a replication-competent HCV virus having similar properties to the JFH-1 virus. Thus, the mutations produce an HCV virus which is able to give rise to infectious virus particles, and accordingly further rounds of viral replication. The HCV virus is typically replication competent in the Huh 7.5 human hepatoma cell line. Alternative cell lines include human Huh 7 human hepatoma cells. It may also be possible to demonstrate replication in human HepG2 cells suitably supplemented with the micro RNA necessary for HCV replication (miR-122).

The HCV virus may produce about 10% or more, preferably 20%, 30%, 40%, 50%, 60%, 70%, more preferably 80%, 90%, 95% or more of the amount of infectious viral particles produced by the JFH-1 virus.

The following Examples illustrate the invention.

### EXAMPLES

### Example 1

### Involvement of the SL9266/PK and the NS5B Polypeptide in HCV Translation

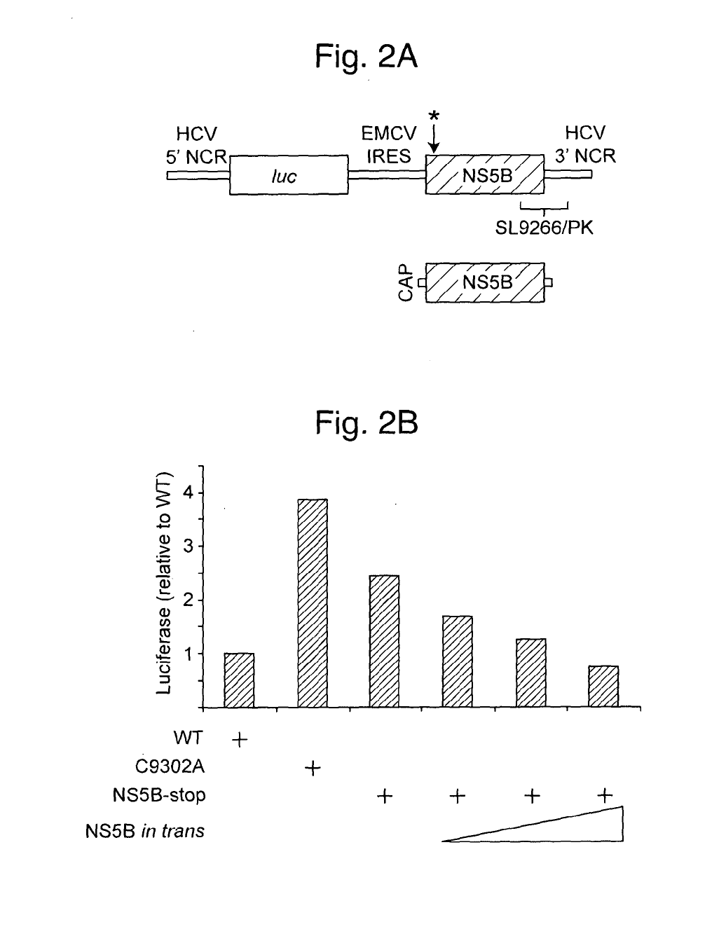

FIG. 2a illustrates two RNA molecules. The top consists of an HCV IRES, a luciferase reporter gene, and EMCV IRES, the coding region for NS5B and the 3′ non-coding region of HCV (the bicistronic reporter). The position of the SL9266/PK RNA structure is indicated. The asterisk indicates the position of a stop codon introduced in certain RNA molecules to prevent synthesis of NS5B. The lower RNA encodes NS5B only, translated from an in vitro transcribed and capped RNA. The translation results are shown in the lower panel. 1 microgram of the bicistronic reporter was transfected into Huh 7.5 cells and the luciferase activity (determined using commercial kit) was quantified 24 hours post-transfection (column 1). A similar amount of bicistronic RNA bearing a C9302A mutation within the sub-terminal bulge loop of SL9266 was transfected into fresh cells in parallel and the luciferase activity determined at 24 hours. This level of expression was normalised to expression from the unmodified template (column 2). Similarly, luciferase activity from a bicistronic template with a stop codon (NS5B-stop) was quantified and normalised to the unmodified bicistronic template (column 3). Co-transfection of 1 microgram of NS5B-stop and increasing levels of capped NS5B RNA (columns 4-6—representing 1, 5 and 10 fold molar excess with regard to the bicistronic reporter) were used to determine the effect of introduction of NS5B in trans, again with the results being normalised to the level of expression of the unmodified bicistronic template.

A bicistronic reporter system based on the Con1b HCV sequence was constructed in order to investigate a possible role for SL9266/PK and NS5B in HCV translation, The sequence of this reporter construct is shown in SEQ ID NO: 3. In this construct, a luciferase reporter coding sequence is under the control of an HCV IRES. Luciferase activity was monitored to determine translation activity in Huh-7.5 cells.

FIG. 2 shows that SL9266/PK and NS5B influenced the HCV IRES-mediated translation of the luciferase reporter gene over a 2-4 fold range in the bicistronic system.

Disruption of the upstream interaction involving the SL9266 stemloop (using mutations C9302A, or G9110U (data not shown) were found to enhance translation. Additionally, premature truncation (NS5B-stop) of the encoded NS5B polypeptide was also found to enhance translation. In contrast, over-expression of NS5B in trans repressed translation.

The results were consistent with a role for NS5B and SL9266/PK in translational control of HCV. Preliminary observations (data not shown) did not support a direct and specific interaction of NS5B and SL9266/PK-containing sequences by electrophoretic mobility shift assays (EMSA) or RNA affinity chromatography (RAC). Accordingly, it is likely that repression of translation exerted by NS5B is indirect. There are a number of scenarios by which this could be achieved; NS5B could sequester or modify a cellular protein required for translation thereby preventing it binding SL9266/PK (or a distal region of the mRNA influenced by SL9266/PK structure—see WP3), or a complex containing a cellular protein and NS5B might exert control following recruitment to SL9266/PK.

These and other possibilities can be analysed using the bicistronic system. Similarly, the reporter system allows for testing of compounds for a modulatory effect on HCV translation.

### Example 2

### Investigation of Interactions of SL9266 in Different HCV Virus Backgrounds

### A. Materials and Methods

Stem-Loop Nomenclature

We use the standardised system for numbering HCV sequences in which stem-loops are designated by the position of the first 5′ paired nucleotide in the structure (Kuiken et al., 2006) with reference to the H77 complete genome sequence (GenBank Accession # AF011753). Stem-loop structures previously designated as 5BSL3.1-3.3), SLIV-VII or SL8828, SL8926, SL9011, SL9061 and SL9118 are, respectively, referred to as SL9033, SL9132, SL9217, SL9266 and SL9324. The 5′ NCR stem-loop IIId is designated SL253 and the three structures that together form the X-tail (5′-SLIII, SLII and SLI-3′) are designated SL9548, SL9571 and SL9601.

Cell Culture

Monolayers of the human hepatoma cell line Huh 7.5 were maintained in Dulbecco's modified minimal essential medium supplemented with 10% (v/v) fetal bovine serum (Invitrogen), 1% non-essential amino acids, 2 mM L-glutamine and 100 U penicillin/100 μg streptomycin/ml (DMEM P/S). Cells were passaged after trypsin/EDTA treatment and seeded at dilutions of 1:3 to 1:5.

HCV cDNA Plasmids and Mutagenesis

The parental firefly luciferase-encoding Con1b replicon—designated pFKnt341-sp-P1-lucEI3420-9605/5.1 (for convenience designated here as Con1b-luc-rep) has been previously reported (Friebe et al., J. Virol., 12047-12057: 2001). Mutations were introduced to the unique Spe I-Xho I sub-fragment of this plasmid cloned in pBluescript II SK (+) (Stratagene) using the SatageneQuickChange™ system. Their presence was then confirmed by DNA sequencing, before rebuilding into the parental plasmid.

The parental plasmid pFK-J6/JFH-1-C-846 (for convenience designated here as J6/JFH-1) full length cDNA has also previously been fully described (B. Lindenbach et at 2005). QuickChange™ mutagenesis was performed on the unique Hind III-Ssp I (8208-10128) fragment sub-cloned in pUC18, confirmed by DNA sequencing and the modified region rebuilt into the parental cDNA on a Hind III-Mlu I fragment (8208-9903).

Replication-incompetent derivatives of both the replicon and infectious virus systems were generated by substitution (GDD to GND) within the active site of the NS5B polymerase.

In Vitro RNA Transcription

1 μg of either J6/JFH derived plasmid cDNA—which includes a 5′ cis acting RNA cleavage ribozyme—or ScaI-linearized Con1b-luc-rep cDNA was used as a template for the production of RNA in vitro using a T7 MEGAscript kit (Ambion) according to the manufacturers' instructions. RNA was purified with an RNeasy mini-kit (Qiagen), the integrity confirmed by denaturing agarose gel electrophoresis and quantified by NanoDrop spectroscopy.

RNA Modification for SHAPE

Templates for SHAPE reactions—either 40 pmol of a sub-genomic RNA transcript (nt. 9005 to the 3′ terminus of the HCV genome) or 10 pmol of full length pFK-J6/JFH-1-C-846 or Con1b-luc-rep RNA transcripts in 10 μl 0.5×Tris-EDTA (pH8.0)(TE)—were heated at 95° C. for 3 min, incubated for 3 mins on ice before being supplemented with 6 μl of folding buffer (330 mM HEPES, [pH 8.0], 20 mM MgCl₂, 330 mMNaCl) and allowed to refold at 37° C. for 20 min.

Samples were then divided in half and incubated with either 1 μl of 100 mM NMIA dissolved in DMSO or 1 μl of DMSO for 45 min at 37° C. Each reaction was terminated by ethanol precipitation following the addition of 100 μl of EDTA (100 mM), 4 μl of NaCl (5M) and 2 μl of glycogen (20 mg/ml). Samples were re-suspended in 0.5×TE containing RNA secure (Ambion) and heated to 65° C. for 10 min before use in the primer extension reaction.

5′-[32P]-Primer Labelling

60 μM of primer was incubated with 10 units of T4 polynucleotide kinase (New England Biolabs), 2 μl of supplied 10× buffer and 12.5 μl γ-[32P]-CTP (PerkinElmer) at 37° C. for 20 min and stopped by incubation at 65° C. for 20 min. Radiolabelled primers were purified trough a 20% PAGE gel (7M urea), passively eluted overnight from the gel slice into water, ethanol precipitated and re-suspended in 100 μl 1 mM HEPES (pH 8.0) before use.

Primer Extension Reactions for SHAPE

NMIA- or control-treated RNA was mixed with 3 μl of 30 μM radiolabelled primer, denatured at 95° C. for 5 min then incubated at 35° C. for 5 min to anneal the primer prior to chilling on ice for 2 min. 6 μl of RT mix was added (5× Superscript III buffer, 17 mM DTT and 1.7 mM dNTPs; Invitrogen), then the sample was incubated at 55° C. for 1 min before the addition of 1 μl of Superscript III (Invitrogen) and continued incubation at 55° C. for a further 30 min.

The DNA template was degraded by the addition of 1 μl of 4M NaOH and incubation at 95° C. for 5 min before the addition of 29 μl of acid stop mix (160 mM un-buffered Tris-HCL, 73% formamide, 0.43×TBE, 43 mM EDTA [pH 8.0], bromophenol blue and xylene cyanol tracking dyes) followed by a further 5 min at 95° C.

Dideoxy nucleotide triphosphate (ddNTP) sequencing markers were generated by the extension of unmodified RNA after the addition of 2 μl of 20 mM ddNTP (Fermentas) prior to the addition of the RT mix. The cDNA extension products were separated by denaturing electrophoresis (7% (19:1) acrylamide:bisacrylamide, 1×TBE, 7M UREA) at 70 W for 1 and 5 hours (the duration dependent on the fragment sizes being analysed) and visualised on a phosphoimager (Fujitsu). Images were analysed for average band intensity/pixel in the NMIA- and DMSO control-reactions at every nucleotide position usingTotalLabID gel analysis software.

Replicon Analysis, Virus Recovery and Quantification

Huh 7.5 cells were transfected by electroporation. Briefly, trypsinized, washed Huh 7.5 cells were re-suspended in phosphate buffered saline (PBS) at 3×10⁷ cells/ml, mixed with 5 μg of in vitro transcribed RNA in a pre-chilled 4 mm cuvette, pulsed once (square wave pulse, 250 V for 25 milliseconds) using a Bio-Rad Gene Pulser Xcell unit, before incubation on ice for 5 min and final resuspension in 10 ml of DMEM P/S.

Luciferase expression by the Con1b-luc-rep replicon was determined at 4, 24, 48 and 72 hours post-transfection from 2.5 ml of transfected re-suspended cells transferred to a six-well plate, washed twice with PBS, lysed with 0.5 ml Glo-Lysis Buffer (Promega) and stored frozen prior to analysis using Bright-Glo lysis buffer (Promega) and a Turner TL-20 luminometer.

For analysis of infectivity of the J6/JFH-1 cDNA, 2 ml of re-suspended transfected cells were transferred to a single well in a six-well plate and the remaining 8 ml transferred to a T75 flask. After three days, monolayers in the six-well plate were washed twice with PBS, fixed with 1 ml 4% paraformaldehyde for 20 min and washed twice again in PBS. Supernatant from the T75 flask was harvested, clarified by centrifugation, filtered through a 0.20 μM filter and the virus titre assayed in triplicate on Huh 7.5 cells and expressed as focus forming units/ml (ffu/ml).

In both instances, replicating virus was assayed by immunofluorescence in fixed cells, permeabilised by incubation in 0.1% Triton PBS for 7 min with constant agitation and subsequently washed with PBS, using a polyclonal sheep antibody to NS5A (αNS5A) diluted 1:5000 in 10% foetal bovine serum (FBS). After incubation for 1 hr. the primary antibody was detected using an AlexaFluor594-conjugated secondary anti-sheep antibody (1:500 in 10% FBS; Invitrogen), washed in PBS and stored under PBS containing 0.1% VECTASHIELD DAPI (Vector Laboratories) before analysis by UV microscopy.

### B. Results

SHAPE Mapping of SL9266

SHAPE (selective 2′-hydroxyl acylation analysed by primer extension) analysis uses chemical modification of the unpaired bases in a folded RNA molecule to render them uncopyable during a primer extension reaction. As a consequence, by judicious choice of primer-binding sites, it is possible to map both local and long-range interactions in an RNA molecule. Additionally, by analysis of individual or compensatory point mutations it is possible to determine the influence on RNA structure of mutations that—in a replicating genome—exert a phenotypic effect.

We investigated the structure of SL9266 by SHAPE analysis of RNA transcripts derived from the luciferase-encoding Con-1b-derived sub-genomic replicon (Con1b-luc-rep) or from J6/JFH-1. In each instance two positive-sense transcripts were used for SHAPE mapping studies; a full-length transcript generated from the bacteriophage T7 polymerase promoter in the plasmid vector or a sub-genomic transcript generated from a PCR product spanning nucleotides 9005 to the extreme 3′ end of the genome.

Without exception, for either Con1b-luc-rep or J6/JFH-1, the results obtained with the longer template were indistinguishable through the analysed regions implying that RNA structures present in the shorter NS5B-3′UTR template are not fundamentally influenced by sequences elsewhere in the virus genome.

We initially analysed the exposure to the chemical modifier (NMIA) of sequences in and around SL9266 in J6/JFH-1. We observed a good correlation between the bioinformatically predicted structure and the previous biochemical mapping of the region (Tuplin et al., 2004). For example, the terminal nucleotides (9239-9242) of the immediately 5′ adjacent structure (SL9118) were unpaired as indicated by the strong terminations at U9240, U9241, A9242 and U9243 (note that when interpreting SHAPE autoradiographs the primer extension product terminates at the base before the uncopyable, chemically acylated, exposed nucleotide). Within the 3′ side of the SL9118 duplex there was an additional obvious termination at U9259 which indicates that G9258 is, as predicted, unpaired.

The sequence immediately 3′ to SL9266 was strongly NMIA-reactive indicating that this region (nucleotides 9313-9323) is predominantly unpaired, as predicted. Also shown is the 5′ portion of the stem of SL9324 (nucleotides 9324-9336) which show no NMIA reactivity, in agreement with the structure predicted (Tuplin et al., 2004).

Within J6/JFH-1 SL9266 per se we analysed the NMIA reactivity and quantified the exposure of individual nucleotides, after subtraction of the signal in the absence of NMIA, normalised over an extended window spanning the region of interest. This approach allows comparisons to be readily made with analogous regions of other genomes or variants of the same sequence (see below).

Exposed nucleotides score positively. In contrast, nucleotides that are poorly or unreactive to NMIA score as negative values. In J6/JFH-1 SL9266, G9273 was well-exposed as were UC9312-9313. The former nucleotide occupies the top of the lower duplex, opposite the sub-terminal bulge loop, whereas the latter are at the 3′ base of the structure.

If the terminal and sub-terminal loops of SL9266 were wholly unpaired we would expect extensive NMIA reactivity of these sequences, respectively occupying nucleotides 9280-9291 and 9298-9305. However, this was not what was observed. Both regions exhibited some exposed nucleotides (9281-9284 and 9298-9300), with the remainder showing lower than average exposure, indicating they were protected from reacting with the NMIA.

It is notable that the regions of the SL9266 terminal and sub-terminal loops that were predominantly NMIA unreactive were those predicted to form long-range interactions with sequences elsewhere in the HCV genome. The remaining regions of J6/JFH-1 SL9266 exhibited lower than average NMIA reactivity, supporting bioinformatic predictions for the duplexed regions.

For comparison, we conducted the same analysis on the Con1b SL9266. Unsurprisingly G9313 and C9266, which form a complementary pair at the base of the lower duplex, were both poorly NMIA reactive. In contrast to the situation with SL9266 of J6/JFH-1 almost every nucleotide in the terminal loop was well exposed (9281-9289), with little or no reactivity in the sub-terminal loop region and only a weak signal for G9273. It was clear from this preliminary comparison that our SHAPE analysis supported the core local duplexed structure of SL9266 in J6/JFH-1 and Con1b, but that the interactions of SL9266—manifest as the exposure of the terminal and sub-terminal loops—with other regions differed significantly.

SHAPE Mapping of Mutants that Influence the Predicted Interactions of SL9266

The preliminary analysis of the native structures of SL9266 in J6/JFH-1 and Con1b prompted us to investigate the consequences for the structure and interactions of SL9266 of mutations that inhibited genome replication. Additionally, we investigated the local RNA structure in the distal regions—centred on nucleotide 9110 and in the X-tail stem-loop SL9571—with which SL9266 is known or predicted to interact. Specifically we analysed the influence of mutations that disrupted (G9110U or C9302A; data not shown) or restored (G9110U and C9302A; data not shown) the proposed interaction between the sub-terminal bulge loop and sequences around nucleotide 9110 (Diviney et al., 2008).

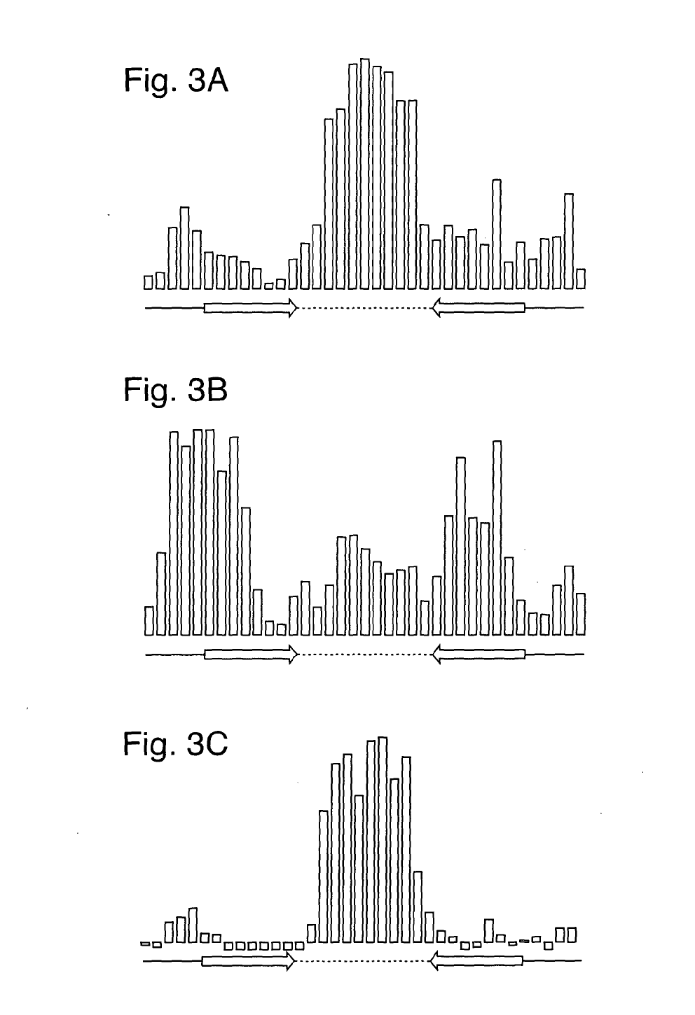

We also investigated the disruption (G9583A; panel A FIG. 3) and restoration (C9287U and G9583A; data not shown) of the interaction of the terminal loop of SL9266 with SL9571 (Friebe et al., 2005).

J6/JFH-1: SL9266 Sub-Terminal Bulgeloop and 9110 Region

In J6/JFH-1 substitutions of G9110U or C9302A were almost indistinguishable in their effect on SL9266 or the SL9266 interacting sequences. Either substitution markedly increased the exposure of sequences in the sub-terminal bulge loop of SL9266 (9298-9305) and simultaneously increased exposure of the sequences (9108-9113) in the upstream regioncentred on nucleotide 9110.

The significantly reduced exposure of G9273—the closing nucleotide at the top of the SL9266 lower duplex, located opposite the sub-terminal bulge loop (FIG. 1A)—suggests that there were additional structural consequences for SL9266 resulting from inhibition of the long-range upstream interaction.

Inhibition of the upstream interaction had little if any influence on the exposure of the terminal loop sequences of SL9266 that interact with SL9571, though there was a minor decrease in reactivity of UU9281-9282 immediately adjacent to the SL9571 interacting sequences. Introduction of the C9302A and G9110U covariant substitutions together did not restore the structure of J6/JFH-1 SL9266 to that seen in the native sequence. Indeed, there was little difference between the structure of SL9266 in the presence of the double mutant or either mutation individually. Likewise, the NMIA reactivity of both SL9571 and the 9110 region in the double mutant was largely indistinguishable from either mutant alone.

Con1b: SL9266 Sub-Terminal Bulgeloop and 9110 Region

There were marked differences between the NMIA-reactivity of SL9266 sequences in Con1b and J6/JFH-1 templates. In Con1b, the native structure of SL9266 appears to predominantly form the upstream interaction, with the terminal loop of SL9266—and the complementary sequences in SL9571—largely unpaired.

Substitutions designed to inhibit the upstream interaction (G9910U or C9302A) increased exposure of sequences within the sub-terminal loop of SL9266, though this was most marked in nucleotides 9298-9300. At the same time the upstream region—including all the proposed interacting sequences—became markedly more NMIA-reactive. The NMIA-reactivity of the terminal and sub-terminal loops of SL9266, and the region around nucleotide 9110, was restored to wild-type levels in the presence of both G9910U or C9302A covariant substitutions. Both the G9110U or C9302A substitutions also resulted in slightly reduced exposure of the terminal loop of SL9266 and sequences in SL9571.

This implies that interactions of Con1b SL9266, although strongly biased in favour of the upstream region, can occur between the terminal loops of SL9266 and SL9571. Although the presence of both substitutions restored near-native NMIA reactivity to the terminal loop of SL9266, there was little apparent change in the exposure of SL9571, though it should be noted that since this region was already largely unpaired and exposed, small changes were difficult to quantify.

J6/JFH-1: SL9266 Terminal Loop and SL9571

We investigated the consequences of disrupting the interaction of the terminal loops of SL9266 and SL9571 by introducing a G9583A substitution to SL9571 in the 3′ X-tail. The NMIA-reactivity observed supported the proposed interaction of these regions; nucleotides 9280-9289 in SL9266 and 9581-9588 in SL9571 became significantly more exposed to NMIA in the presence of G9583A. Within SL9571, the regions flanking the terminal loop exhibited reduced NMIA reactivity. Conversely, there was little or no change in the exposure of sequences in the 9110 region.

Restoration of the interaction of the J6/JFH-1 SL9571 and SL9266 terminal loops, by introduction of the covariant substitutions of C9287U and G9583A reduced the exposure of both terminal loop regions and increased the exposure of sequences flanking the terminal loop of SL9571 to near-native levels.

The striking changes in the structure of SL9571 between that in an unmodified template and in one bearing a G9583A mutation are particularly obvious in the raw SHAPE reactivity; in the former the region interacting with SL9266 is less NMIA-exposed than the flanking sequences that occupy the proposed duplex region of SL9571. In contrast, this exposure is reversed in the G9583A mutant, where the terminal loop of SL9571 is the only region that exhibits a high level of SHAPE reactivity. Neither the G9583A mutation alone, or the combination of C9287U and G9583A, resulted in significant changes to the NMIA accessibility of sequences in the region around nucleotide 9110.

Con1b: SL9266 Terminal Loop and SL9571

Substitutions of C9287U alone, or C9287U and G9583A, designed to test the interaction of SL9571 and SL9266 had little effect on the exposure of the terminal loop of the latter structure. In addition, these substitutions had no measurable effect on the remainder of SL9266 or on the upstream region around 9110 with the exception of a reduction in exposure of G9103 in both modified templates (which was also seen in all mutagenized templates. However, C9287U did marginally increase the exposure of the terminal loop of SL9571, particularly when compared with sequences that flank the terminal loop. These changes were at least partially restored by the presence of both C9287U and G9583A mutations.

Phenotypic Consequences of Mutagenesis of the J6/JFH-1 SL9266 Pseudoknot

We have previously reported genetic evidence supporting a predicted interaction between a nucleotide sequences centred on nucleotides 9110 and 9302 in Con1b-based sub-genomic replicon studies (Diviney et al., 2008). Mutations that disrupted the bioinformatically-predicted pairing between the upstream region and the sub-terminal bulge loop of SL9266 inhibited replication. Introduction of covariant changes at 9110 and 9302 restored replication, thereby confirming the importance of the RNA-RNA interaction, rather than the sequence per se.

We reasoned that, since the long-range interaction between 9110 and 9302 is equally well-predicted in the genotype 2a JFH-1 genome—indeed the predicted interacting sequences are identical—analogous mutations built into the J6/JFH-1 HCVcc system would exhibit equivalent phenotypes.

The J6/JFH-1 genome was modified by introduction of single substitutions of G9110U or a double substitution of C9302A and C9303G, all of which were predicted to inhibit the interaction of the sub-terminal bulge loop of SL9266 and the upstream region. Additionally we engineered both the G9110U and C9302A mutations into the same genome, restoring complementarity between the sequences.

In vitro transcribed RNA was transfected into Huh-7.5 cells and supernatant virus was harvested and quantified 72 hours later. In these studies the control unmodified J6/JFH-1 genome generated ˜105 focus forming units per ml (ffu/ml), whereas a genome with a mutation within the active site of the NS5B polymerase did not replicate and generated no progeny virus.

Genomes bearing a G9110U substitution replicated indistinguishably from the unmodified virus whereas genomes carrying C9302A, C9302A and C9303G or G9110U and C9302A all yielded approximately 0.5 log 10 less virus than the positive control. Further passage of the these viruses did not increase virus yield.

Because of the striking divergence of the observed virus phenotype from those expected from previous replicon-based studies we went on to investigate the consequences of mutagenesis of the core structure of J6/JFH-1 SL9266, or sequences implicated in the interaction of SL9266 with sequences in the X-tail.

The double substitution of C9275G and G9293A, both of which disrupt the upper duplexed region of SL9266, did not yield detectable virus upon RNA transfection but, with further passage, generated ˜102 ffu/ml presumably reflecting the selection of revertant or covariant genomes with a restored ability to replicate. The inclusion of additional substitutions of C9278U and G9296C to a genome already bearing C9275G and G9293A, thereby restoring the integrity of the upper duplex of SL9266, completely restored the ability to replicate at parental J6/JFH-1 levels.

Finally, a single point mutation of G9583A in the terminal loop of SL9571 significantly reduced virus yield which was only partially restored upon subsequent serial passage. In agreement with published studies (Lohmann et al., 2003) indicating that this region binds to the terminal loop of SL9266, we demonstrated that the double mutant C9287U and G9583A replicated at wild-type levels.

Conclusions

To date, the two widely accepted and exploited approaches to study replication of HCV are genotype 1b-derived sub-genomic replicons or a genotype 2a JFH-1 (or chimeric derivatives thereof) HCVcc system (Lohmann et al., 1999; Wakita et al., 2005).

To replicate efficiently, the genome of the former has acquired adaptive mutations, predominantly in the NS5A non-structural protein, during in vitro passage (Blight et al., 2000; Lohmann et al., 2003; Lohmann et al., 2001), These enhance genome replication but may be incompatible with in vivo replication (Bukh et al., 2002), or do not increase the yield of viral particles (Ikeda et al., 2002; Pietschmann et al., 2002).

In contrast, JFH-1, derived from a fulminant hepatitis case, both replicates well and yields high levels of infectious progeny particles (in vitro and in vivo) without acquiring adaptive mutations in cell culture (Lindenbach et al., 2005; Wakita et al., 2005; Zhong et al., 2005).

We found that the differences in replication-competence of the two HCV genotypes could be explained at the level of the structure and interactions present in the SL9266/PK.

Despite the similarities in the core structure of SL9266 there were striking differences in the interactions exhibited by the terminal and sub-terminal loops when the two genotypes were compared. These differences were obvious in the analysis of unmodified templates and were subsequently supported by mutagenesis of SL9266 and the regions with which they interact. Significantly, the previously reported ‘kissing loop’ long-range interaction between the terminal loop of SL9266 and SL9571 also influenced the structure of the latter region of the X-tail.

The most marked differences in the interactions observed in Con1b and JFH-1 were between the terminal loop of SL9266 and sequences that form the terminal loop of SL9571. As already indicated, in JFH-1 this interaction was readily observable, was disrupted by substitution of G9583A, and was fully restored by the addition of a covariant C9287U mutation. In contrast, in Con1b there was little evidence for an interaction between these regions of the genome using SHAPE mapping. The G9583A substitution in Con1b did not increase exposure of the terminal loop of SL9266 and nor did it change the NMIA accessibility of sequences in the X-tail region. We therefore propose that the steady-state conformation and interactions of SL9266 differs fundamentally between the Con1b and JFH-1 genomes.

A striking and unexpected observation resulting from the comparison of native and mutagenized structures of SL9266 is that the interaction between the terminal loop of SL9266 and sequences in the 3′ X-tail markedly influences the structure of SL9571. In J6/JFH-1 the unmodified template exhibits approximately 2-fold greater NMIA-reactivity in the sequences that are predicted to form the stem region of SL9571. In contrast, in the presence of G9583A, the predicted stem region of SL9571 becomes NMIA-unreactive, whereas the entire terminal loop (9581-9588) becomes exposed

This situation is very similar in the unmodified structure of Con1b SL9571, in which the vast majority of SL9266 pairing is with the 9110 region, a situation exacerbated by the presence of the G9583A substitution which abrogates NMIA-reactivity of the sequences flanking the terminal loop of SL9571. Our preliminary studies have indicated that SL9266 of H77, the genotype 1a prototype strain, is similar in structure to Con1b (data not shown), with the terminal loop of SL9266 being unpaired to SL9571 and the sub-terminal loop being fully paired with sequences around nucleotide 9110.

We conclude that the sequences that contribute to the formation of SL9571—nucleotides 9571-9597—only form a stem-loop structure when the ‘kissing loop’ interaction with SL9266 does not occur.

Our data show that the upstream interaction of Con1b SL9266 inhibits interaction of the latter with SL9571. In contrast, in J6/JFH-1 the 5′ and 3′ interactions are independent. Additionally we show that the ‘kissing loop’ interaction fundamentally influences the structure of SL9571 in J6/JFH-1, and that similar—albeit less marked—changes are discernible in Con1b SL9571.

We propose that one function of SL9266 is to control the structure of SL9571 via this ‘kissing loop’ interaction. Since the ‘kissing loop’ interaction has been shown to be essential in both Con1b and J6/JFH-1, it suggests that SL9571 is likely involved in a critical replication phase preceding the encapsidation event.

### Example 3

### Experimental Systems

Translation Assay

The translation assay uses the bicistronic reporter gene system described in Example 1. In brief, a firefly luciferase reporter gene is under the translational control of the HCV internal ribosome entry site (IRES) as part of the complete 5′ untranslated region of HCV. The luciferase gene is followed by an IRES from an unrelated virus (encephalomyocarditis virus; EMCV) which drives expression of the RNA dependent RNA polymerase (RdRp) of HCV (the NS5B protein) which, in turn, is followed by the HCV 3′ untranslated region (3′ UTR).

All experiments are routinely conducted in human hepatoma cells—Huh-7.5. This is the cell line in which HCV replicates. However, we have also investigated the bicistronic reporter system in HepG2, HeLa and murine cell lines, as well as in cell free translation systems supplemented with Huh-7.5 cell extracts. With the exception of murine cell lines we demonstrated translational control in all the cell lines tested, and in a cell free system.

The readout of the translation assay is luciferase activity. To control for differences in transfection we co-transfected a separate plasmid encoding renilla luciferase and normalize all firefly results to this.

The translation inhibition assay is typically conducted by co-transfecting RNA generated in vitro from the bicistronic reporter plasmid with oligonucleotides to be tested. Assays for luciferase are routinely conducted four hours post transfection.

Sub-Genomic Replicon Assay

This assay uses a sub-genomic replicon analogous to that described by Lohmann (Lohmann et al., 1999). As above, we co-transfect an RNA encoding renilla firefly luciferase to allow normalization of transfection levels. In these assays we usually transfect the sub-genomic replicon (and renilla) RNA 24 hours before addition of the oligonucleotides.

Virus Production Assay

This assay uses the HCVcc system that measures the production of infectious genotype 2a JFH-1 hepatitis C virus as described previously (Wakita et al., 2005). In this assay oligonucleotides directed against RNA sequences or structures are transfected into Huh-7.5 cells and a known amount of virus added 4 hours later. After 24 hours incubation the cell sheet is fixed and virus replication quantified by immunofocal staining for virus antigen (in this instance the NS5A protein). Infected cells are counted using a fluorescence microscope.

Oligonucleotides

Synthetic oligonucleotides used in our studies were produced by Invitrogen and are LNA (locked nucleic acids). This means they have modified chemistry that renders them less easily degraded in the cell.

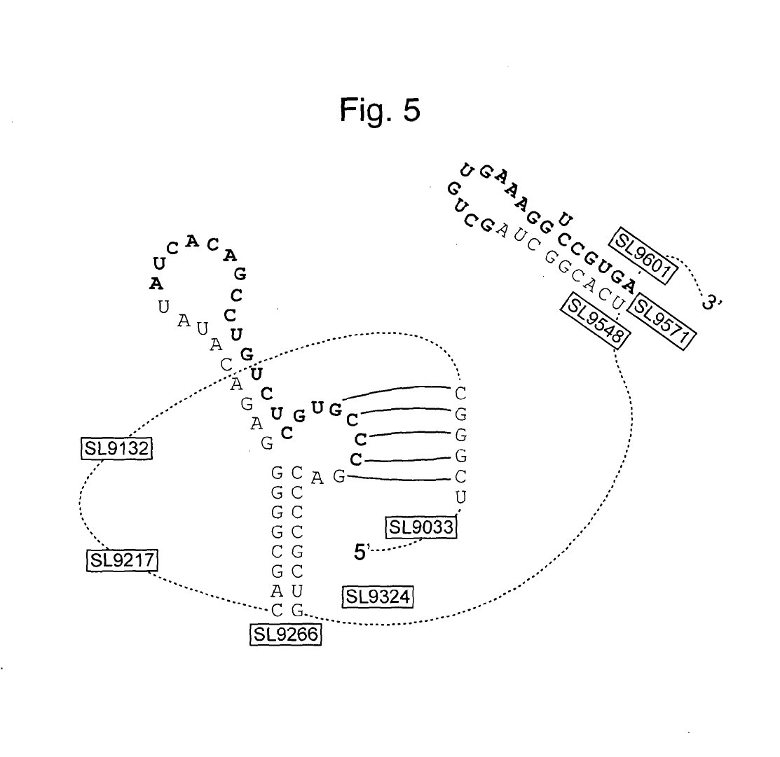

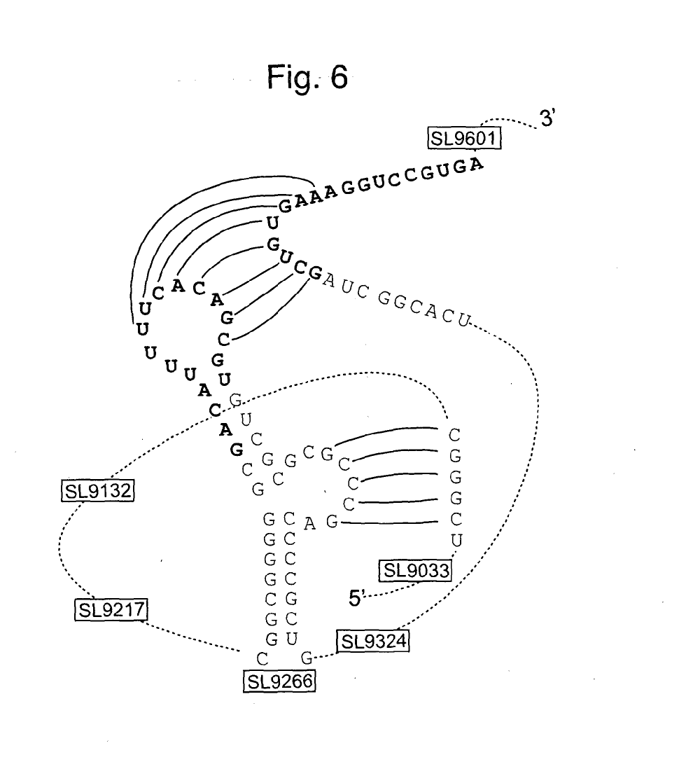

We have tested four oligos and relevant controls directed against either SL9266 or SL9571 (the stemloop in the 3′ UTR with which SL9266 forms a “kissing loop”). The oligos directed against Con1b SL9266 and SL9571 are illustrated in FIG. 5 and below, those against SL9266 and SL9571 of JFH-1 are illustrated in FIG. 6 and below. The oligonucleotide sequences are shown with the LNA-modified bases underlined. A variety of physical characteristics of the oligo are listed. The HCV sequences complementary to the oligonucleotides are illustrated highlighted in bold in the graphic.

Con1b anti-SL9266 LNA oligonucleotide
5′-GGGCACGAGACAGGCTGTGAT-3′
21 bases
Tₘ as RNA/DNA duplex = 84° C.
Self hybridization score 29
Secondary structure score 22
Randomised LNA oligonucleotide
5′-GCACAGCGCAAGTATGTTA-3′
19 bases
Tₘ as RNA/DNA duplex = 85° C.
Self hybridization score 37
Secondary structure score 23
JFH-1 anti-SL9266 LNA oligonucleotide
5′-ACGCTGTGAAAAATGTC-3′
17 bases
Tₘ as RNA/DNA duplex = 79° C.
Self hybridization score 37
Secondary structure score 30
Con1b/JFH-1 anti-SL9571 LNA oligonucleotide (this
oligonucleotide targets a conserved sequence in
both the Con1b and JFH-1 isolates)
5′-TCACGGACCTTTCACAGC-3′
18 bases
Tₘ as RNA/DNA duplex = 85° C.
Self hybridization score 28
Secondary structure score 24

### Results

Translation Assay

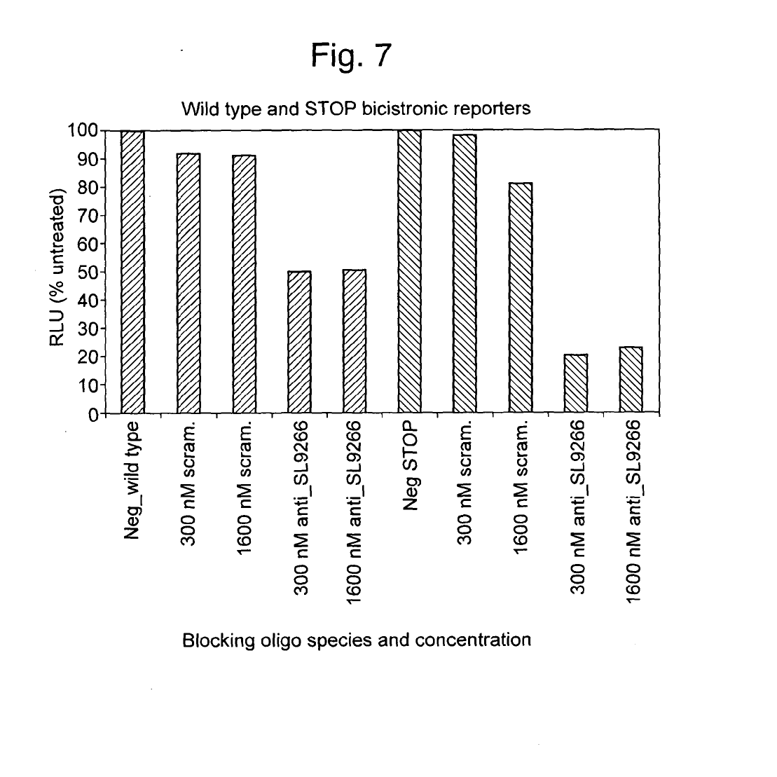

Oligonucleotides directed against SL9266 inhibit translation of hepatitis C virus. Results are shown normalized to untreated samples. A scrambled sequence (negative) control has only a limited influence, reaching a maximum of ˜20% inhibition when added at 1600 nM. In contrast, oligos directed against SL9266 exhibit up to 80% inhibition of translation. In these assays maximum inhibition is observed in the version of the bicistronic reporter that does not synthesise NS5B (see FIG. 7).

Replication Assay Using the Sub-Genomic Replicon

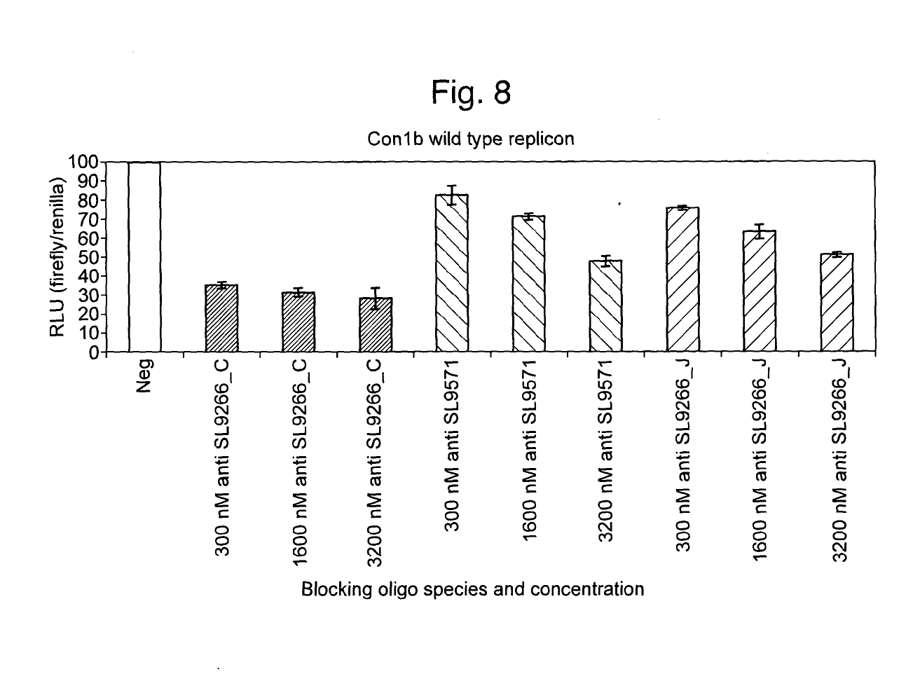

In this assay we investigated inhibition of luciferase gene activity encoding by the sub-genomic replicon and the influence of prior transfection of oligonucleotides directed against SL9266 or SL9571. The sub-genomic replicon is a genotype 1b sequences (Con1b) and directly complementary oligonucleotides directed against SL9266 (labelled anti SL9266_C) inhibit replication by ˜70% (see FIG. 8). In contrast, an oligonucleotide complementary to SL9266 of genotype 2a (JFH-1), is only partially inhibitory. This oligonucleotide exhibits only partial complementarity to the Con1b sub-genomic replicon:

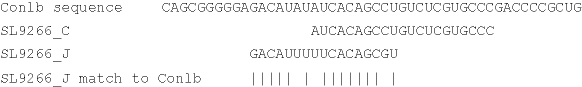

Virus Replication Assay Using JFH-1 (Genotype 2a)

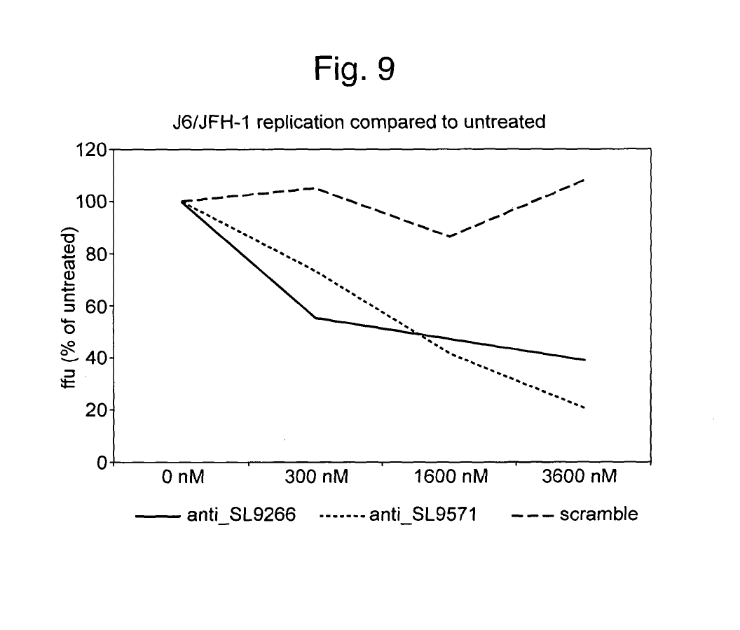

In this assay we have tested oligonucleotides directed against SL9266 and SL9571. Compared to untreated cells the scrambled oligonucleotide has little or no influence on virus replication. In comparison, increasing amounts of oligonucleotide directed against SL9266 or SL9571 inhibit virus replication by up to 60-80%, see FIG. 9.

1. A method for identifying a compound that modulates hepatitis virus C (HCV) translation and/or replication, said method comprising:
(a) contacting an RNA comprising the SL9266/PK pseudoknot or a variant thereof and a translatable reporter coding sequence with the compound in the presence of an NS5B polypeptide or a variant thereof; and
(b) measuring translation of the reporter coding sequence.

2. A method according to claim 1, which further comprises:
(c) comparing the translation of the reporter coding sequence measured in b) with a control value obtained for an RNA of a) that has not been contacted with the compound, and thereby determining whether the compound is a modulator of HCV translation and/or replication.

3. A method according to claim 1, wherein said NS5B polypeptide or variant thereof is expressed in trans.

4. A method according to claim 1, wherein said RNA further comprises a translatable NS5B or NS5B variant coding sequence.

5. A method according to claim 4, wherein the reporter coding sequence and NS5B or NS5B variant coding sequence are translated from different cistrons of the RNA.

6. A method according to claim 1, wherein said reporter coding sequence and/or said NS5B or NS5B variant coding sequence is operably linked to an internal ribosome entry site (IRES).

7. A method according to claim 6, wherein said IRES operably linked to said reporter coding sequence is an HCV IRES.

8. A method according to claim 1, wherein said SL9266/PK pseudoknot is comprised in a 3′ non-coding region derived from HCV and/or said RNA comprises a 5′ non-coding region derived from HCV.

9. A method for identifying a compound that enhances or inhibits repression of HCV translation by the NS5B polypeptide, the method comprising carrying out a method according to claim 1 and thereby identifying a compound that enhances or inhibits repression of HCV translation by the NS5B polypeptide.

10. A method for identifying a compound that increases or decreases HCV replication, the method comprising carrying out a method according to claim 1 and thereby identifying a compound that increases or decreases HCV replication.

11. A method for identifying a compound suitable for the prevention or treatment of HCV infection, the method comprising carrying out a method according to claim 1 and thereby identifying a compound suitable for the prevention or treatment of a disease associated with HCV infection.

12. An RNA comprising the SL9266/PK pseudoknot or a variant thereof and a translatable reporter coding sequence.

13. An RNA according to claim 12, which further comprises a translatable NS5B or NS5B variant coding sequence.

14. An RNA according to claim 13, wherein the reporter coding sequence and NS5B or NS5B variant coding sequence are located on different cistrons.

15. An RNA according to claim 11, wherein said reporter coding sequence and/or said NS5B or NS5B variant coding sequence is operably linked to an internal ribosome entry site (IRES).

16. An RNA according to claim 15, wherein said IRES operably linked to said reporter coding sequence is an HCV IRES.

17. An RNA according to claim 12, wherein said SL9266/PK pseudoknot is comprised in a 3′ non-coding region derived from HCV and/or said RNA comprises a 5′ non-coding region derived from HCV.

18. A modulator of HCV translation and/or replication identified by the method claim 1.

19. (canceled)

20. A method of preventing or treating HCV infection in a subject, comprising administering to the subject an effective amount of a modulator of HCV translation and/or replication of claim 18.

21. (canceled)

22. A method for producing a replication-competent HCV virus, said method comprising:
(a) determining the stability of RNA secondary structures of one or more portions of the genome of the HCV virus;
(b) comparing the stability of said RNA secondary structures with the stability of corresponding structures of the JFH-1 HCV virus; and
(c) introducing mutations into the genome of the HCV virus which stabilise said RNA secondary structures in a similar manner to the corresponding structures of the JFH-1 HCV virus, thereby producing a replication-competent HCV virus.

23. A method according to claim 22, wherein said RNA secondary structures comprise RNA secondary structures from the coding and/or 3′ non-coding region of the genome of the HCV virus.

24. A method according to claim 23, wherein said RNA secondary structures comprise the SL9266/PK pseudoknot.

25. A method according to claim 24, wherein said mutations enhance stability of the apical loop interaction of SL9266/PK and/or decrease stability of the bulge loop interaction of SL9266/PK in said HCV virus.

26. A method according to claim 22, wherein said mutations alter hydrogen bonding in or between said RNA secondary structures.

27. A method according to claim 22, wherein when said mutations are introduced into a coding region of the genome of the HCV virus, they do not impair function of an encoded protein.

28. An oligonucleotide comprising 8 to 48 nucleotides in length, wherein the oligonucleotide is substantially complementary to part or all of the region from 9266 to 9314 of HCV.

29. An oligonucleotide according to claim 28, wherein the oligonucleotide is 8 to 30 nucleotides in length.

30. An oligonucleotide according to claim 28, wherein the oligonucleotide is 100% complementary to part or all of the region from 9266 to 9314 of HCV, or wherein the oligonucleotide includes 1, 2, 3 or 4 mismatches.

31. An oligonucleotide according to claim 28, wherein the oligonucleotide comprises one or more locked nucleic acids.

32. An oligonucleotide according to claim 28, for use in the prevention or treatment of HCV infection.


SEQUENCE LISTING (15 sequences)

SEQ ID NO: 1 - DNA, 1773 bp, Hepatitis C virus
tcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccga

SEQ ID NO: 2 - protein, 591 aa, Hepatitis C virus
SMSYTWTGALITPCAAEETKLPINALSNSLLRHHNLVYATTSRSASLRQKKVTFDRLQVLDDHYRDVLKEMKAKASTVKAKLLSVEEACKLTPPHSARSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKAKKCPMGFAYDTRCFDSTVTENDIRVEESIYQCCDLAPEARQAIRSLTERLYIGGPLTNSKGQNCGYRRCRASGVLTTSCGNTLTCYLKAAAACRAAKLQDCTMLVCGDDLVVICESAGTQEDEASLRAFTEAMTRYSAPPGDPPKPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR

SEQ ID NO: 3 - DNA, 4734 bp, Artificial Sequence
  Bicistronic reporter construct
gagctcgccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaacgggcgcgccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatgggtaagcctatccctaaccctctcctcggtctcgattctacgtcggcgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactagt

SEQ ID NO: 4 - DNA, 48 bp, Hepatitis C virus
atgagcacgaatcctaaacctcaaagaaaaaccaaacgtaacaccaac

SEQ ID NO: 5 - DNA, 18 bp, Artificial Sequence
  Oligonucleotide complementary to SL9571 stem loop
tcacggacctttcacagc

SEQ ID NO: 6 - DNA, 21 bp, Artificial Sequence
  Con1b anti-SL9266 LNA oligonucleotide
nnncncgagncagnctgtnnn

SEQ ID NO: 7 - DNA, 19 bp, Artificial Sequence
  Randomised LNA oligonucleotide
nnncagngcnagtntgnnn

SEQ ID NO: 8 - DNA, 17 bp, Artificial Sequence
  JFH-1 anti-SL9266 LNA oligonucleotide
nnnntgtgnanaatnnn

SEQ ID NO: 9 - DNA, 18 bp, Artificial Sequence
  Con1b/JFH-1 anti-SL9571 LNA oligonucleotide
nnncggnccnttnacnnn

SEQ ID NO: 10 - RNA, 48 nt, Artificial Sequence
  Con1b sequence
cagcgggggagacauauaucacagccugucucgugcccgaccccgcug

SEQ ID NO: 11 - RNA, 21 nt, Artificial Sequence
  SL9266_C oligonucleotide
aucacagccugucucgugccc

SEQ ID NO: 12 - RNA, 17 nt, Artificial Sequence
  SL9266_J oligonucleotide
gacauuuuucacagcgu

SEQ ID NO: 13 - RNA, 27 nt, Artificial Sequence
  Con1b SL9571 stemloop
ucacggcuagcugugaaagguccguga

SEQ ID NO: 14 - RNA, 48 nt, Artificial Sequence
  JFH-1 SL9266 stemloop
cggcgggggcgacauuuuucacagcgugucgcgcgcccgaccccgcuc

SEQ ID NO: 15 - RNA, 47 nt, Artificial Sequence
  JFH-1 SL9266 stemloop
cggcgggggcgacauuuuucacagcgugucgcgcgcccgaccccgcu